(19) United States
(12) Patent Application Publication
(10) Pub. No.: US 2024/0060815 A1
(43) Pub. Date: Feb. 22, 2024

(54) Multidimensional Multivariate Multiple Sensor System

(71) Applicant: Sleep Number Corporation, Minneapolis, MN (US)

(72) Inventors: Steven Jay Young, Los Gatos, CA (US); Carl Hewitt, San Jose, CA (US); Jonathan M. Olson, San Jose, CA (US); Alan Luckow, Ben Lomond, CA (US); Omid Sayadi, San Jose, CA (US)

(21) Appl. No.: 18/240,016

(22) Filed: Aug. 30, 2023

Related U.S. Application Data
(63) Continuation of application No. 17/984,414, filed on Nov. 10, 2022, now abandoned.
Application No. 17/984,414 is a continuation of application No. 16/595,848, filed on Oct. 8, 2019, now abandoned.
(60) Provisional application No. 62/804,623, filed on Feb. 12, 2019.

(51) Int. Cl. G01G 19/44; G01V 9/00; G01G 19/52; G06N 5/04; G06N 20/00; G01G 21/02; A47C 19/22; A61B 5/11; G08B 21/22; A61B 5/00; A47C 19/02; G05B 15/02; A61B 5/0205

(52) U.S. Cl. CPC G01G 19/445; G01V 9/00; G01G 19/52; G06N 5/04; G06N 20/00; G01G 21/02; A47C 19/22; A61B 5/1115; A61B 5/1102; G08B 21/22; A61B 5/6891; A47C 19/027; G05B 15/02; A61B 5/0205; A61B 5/4818; A61B 5/6892; A61B 5/7203; A61B 5/7246; A61B 5/725; A61B 5/7267; A61B 5/7278; A61B 5/7282; A61B 5/7415; A61B 2560/0223; A61B 5/0816

(57) ABSTRACT

Devices and methods for determining item-specific information for single or multiple items on one or multiple substrates are described. The method includes generating multiple sensor multiple dimensions array (MSMDA) data from multiple sensors, where each of the multiple sensors capture sensor data for one or more items in relation to a substrate. For each item, the method includes determining relationships between the multiple sensors based on characteristics of the MSMDA data, determining a location of the item on the substrate based on at least the determined relationships between the multiple sensors, determining an angular orientation of the item on the substrate based on at least the determined relationships between the multiple sensors, and determining a body position of the subject on the substrate based at least the determined relationships between the multiple sensors, the location of the subject, and the angular orientation of the item.

### CROSS-REFERENCE TO RELATED APPLICATIONS

This application is a continuation of U.S. patent application Ser. No. 17/984,414, filed Nov. 10, 2023, which is a continuation of U.S. patent application Ser. No. 16/595,848, filed Oct. 8, 2019, which claims priority to and the benefit of U.S. Provisional Patent Application Ser. No. 62/804,623, filed Feb. 12, 2019, the entire disclosures of which are hereby incorporated by reference.

### TECHNICAL FIELD

This disclosure relates to systems and methods for determining biometric parameters and other person-specific information.

### BACKGROUND

Sensors have been used to detect heart rate, respiration and presence of a single subject using ballistocardiography and the sensing of body movements using noncontact methods but are often not accurate at least due to their inability to adequately distinguish external sources of vibration and distinguish between multiple subjects. In addition, the nature and limitations of various sensing mechanisms make it difficult or impossible to accurately determine a subject's biometrics, presence, weight, location and position on a bed due to factors such as air pressure variations or the inability to detect static signals.

### SUMMARY

Disclosed herein are implementations of devices and methods for employing gravity and motion to determine biometric parameters and other person-specific information for single or multiple subjects at rest and in motion on one or multiple substrates is described. In an implementation, a method for determining item specific parameters includes generating multiple sensor multiple dimensions array (MSMDA) data from multiple sensors, where each of the multiple sensors capture sensor data for one or more items in relation to a substrate, and where an item is a subject or an object. For each identified item, the method includes determining relationships between the multiple sensors based on characteristics of the MSMDA data, determining a location of the item on the substrate based on at least the determined relationships between the multiple sensors, determining an angular orientation of the item on the substrate based on at least the determined relationships between the multiple sensors, and determining a body position of the item on the substrate based at least the determined relationships between the multiple sensors, the location of the subject, and the angular orientation of the item.

### BRIEF DESCRIPTION OF THE DRAWINGS

The disclosure is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity.

FIG. 1 is an illustration of a bed incorporating sensors as disclosed herein.

FIG. 2 is an illustration of the bed frame with sensors incorporated, the bed frame configured to support a single subject.

FIG. 3 is an illustration of a bed frame with sensors incorporated, the bed frame configured to support two subjects.

FIG. 4 is a system architecture for a multidimensional multivariate multiple sensors system.

FIG. 5 is a processing pipeline for obtaining sensors data.

FIG. 6 is a pre-processing pipeline for processing the sensors data into multiple sensors multiple dimensions array (MSMDA) data.

FIG. 7 is a flowchart for determining weight from the MSMDA data.

FIG. 8 is a flowchart for performing spatial analysis using the MSMDA data.

FIG. 9 is a flowchart for performing relationship analysis using the MSMDA data.

FIG. 10 is a flowchart for performing location analysis using the MSMDA data.

FIGS. 11A-D are example surface location maps for a multidimensional multivariate multiple sensors system with 4 sensors.

FIG. 12 is a flowchart for performing orientation analysis using the MSMDA data.

FIGS. 13A-D are example orientation maps.

FIG. 14 is a flowchart for performing position analysis using the MSMDA data.

FIGS. 15A-B are flowcharts for performing spatial analysis using machine learning supervised and unsupervised, respectively.

FIG. 16 is a swim lane diagram for performing spatial analysis using machine learning.

FIGS. 17A-B is a flowchart for detecting bed presence and an example graphical representation.

FIGS. 18A-B is flowchart for detecting bed presence with in/out transitions and an example graphical representation.

FIG. 19 is a swim lane diagram for detecting bed presence using machine learning.

FIG. 20 is a swim lane diagram for generating classifiers for new devices or refreshing classifiers for existing devices.

### DETAILED DESCRIPTION

Disclosed herein are implementations of systems and methods employing gravity and motion to determine biometric parameters and other person-specific information for single or multiple subjects at rest and in motion on one or multiple substrates. The systems and methods use multiple sensors to sense a single subject's or multiple subjects' body motions against the force of gravity on a substrate, including beds, furniture or other objects, and transforms those motions into macro and micro signals. Those signals are further processed and uniquely combined to generate the person-specific data, including information that can be used to further enhance the ability of the sensors to obtain accurate readings. The sensors are connected either with a wire, wirelessly or optically to a host computer or processor which may be on the internet and running artificial intelligence software. The signals from the sensors can be analyzed locally with a locally present processor or the data can be networked by wire or other means to another computer and remote storage that can process and analyze the real-time and/or historical data. In an implementation, an item refers to both subjects and objects, where subjects include persons, animals, mammals, animate beings, and the like, and object includes inanimate things and the like.

The sensors are designed to be placed under, or be built into a substrate, such as a bed, couch, chair, exam table, floor, etc. The sensors can be configured for any type of surface depending on the application. Additional sensors can be added to augment the system, including light sensors, temperature sensors, vibration sensors, motion sensors, infrared sensors, image sensors, video sensors, and sound sensors as non-limiting examples. Each of these sensors can be used to improve accuracy of the overall data as well as provide actions that can be taken based on the data collected. Example actions might be: turning on a light when a subject exits a bed, adjusting the room temperature based on a biometric status, alerting emergency responders based on a biometric status, sending an alert to another alert-based system such as: Alexa®, Google Home® or Siri® for further action.

The data collected by the sensors can be collected for a particular subject for a period of time, or indefinitely, and can be collected in any location, such as at home, at work, in a hospital, nursing home or other medical facility. A limited period of time may be a doctor's visit to assess weight and biometric data or can be for a hospital stay, to determine when a patient needs to be rolled to avoid bed sores, to monitor if the patient might exit the bed without assistance, and to monitor cardiac signals for atrial fibrillation patterns. Messages can be sent to family and caregivers and/or reports can be generated for doctors.

The data collected by the sensors can be collected and analyzed for much longer periods of time, such as years or decades, when the sensors are incorporated into a subject's personal or animal's residential bed. The sensors and associated systems and methods can be transferred from one substrate to another to continue to collect data from a particular subject, such as when a new bed frame is purchased for a residence or retrofitted into an existing bed or furniture.

The highly sensitive, custom designed sensors detect wave patterns of vibration, pressure, force, weight, presence and motion. These signals are then processed using proprietary algorithms which can separate out and track individual source measurements from multiple people, animals or other mobile or immobile objects while on the same substrate.

These measurements are returned in real-time as well as tracked over time. Nothing is attached to the subject. The sensors can be electrically or optically wired to a power source or operate on batteries or use wireless power transfer mechanisms. The sensors and the local processor can power down to zero or a low power state to save battery life when the substrate is not supporting a subject. In addition, the system may power up or turn on after subject presence is detected automatically.

The system is configured based on the number of sensors. Because the system relies on the force of gravity to determine weight, sensors are required at each point where an object bears weight on the ground. For other biometric signals fewer sensors may be sufficient. For example, a bed with four wheels or legs may require a minimum of four sensors, a larger bed with five or six legs may require five or six sensors, and a chair with four legs may require sensors on each leg. The number of sensors is determined by the needed application. The unique advantage of multiple sensors is the ability to map and correlate a subject's weight, position, and bio signals. This is a clear advantage in separating out a patient's individual signals from any other signals as well as combining signals uniquely to augment the signals for a specific biosignal. Additional sensor types can be used to augment the signal, such as light sensors, temperature sensors, accelerometers, vibration sensors, motion sensors, and sound sensors.

The system can be designed to configure itself automatically based on the number of sensors determined on a periodic or event-based procedure. A standard configuration would be four sensors per single bed with four legs to eight leg sensors for a multiple person bed. The system would automatically reconfigure for more or less sensors. Multiple sensors provide the ability to map and correlate a subject's weight, position, and bio signals. This is necessary to separate multiple subjects' individual signals.

Some examples of the types of information that the disclosed systems and methods provide are dynamic center of mass and center of signal locations, accurate bed exit prediction (timing and location of bed exit), the ability to differentiate between two or more bodies on a bed, body position analysis (supine/prone/side), movement vectors for multiple subjects and other objects or animals on the bed, presence, motion, location, angular orientation, direction and rate of movement, respiration rate, respiration condition, heart rate, heart condition, beat to beat variation, instantaneous weight and weight trends, and medical conditions such as heart arrhythmia, sleep apnea, snoring, restless leg, etc. By leveraging multiple sensors that detect the z-axis and other axes of the force vector of gravity, and by discriminating and tracking the center of mass or center of signal of multiple people as they enter and move on a substrate, not only can the disclosed systems and methods determine presence, motion and cardiac and respiratory signals for multiple people, but they can enhance the signals of a single person or multiple people on the substrate by applying the knowledge of location to the signal received. Secondary processing can also be used to identify multiple people on the same substrate, to provide individual sets of metrics for them, and to enhance the accuracy and strength of signals for a single person or multiple people. For example, the system can discriminate between signals from an animal jumping on a bed, another person sitting on the bed, or another person lying in bed, situations that would otherwise render the signal data mixed. Accuracy is increased by processing signals differently by evaluating how to combine or subtract signal components from each sensor for a particular subject.

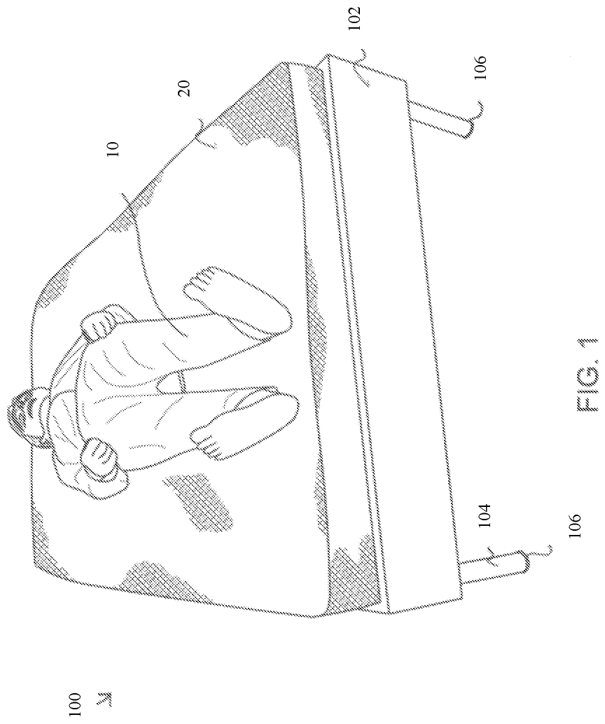

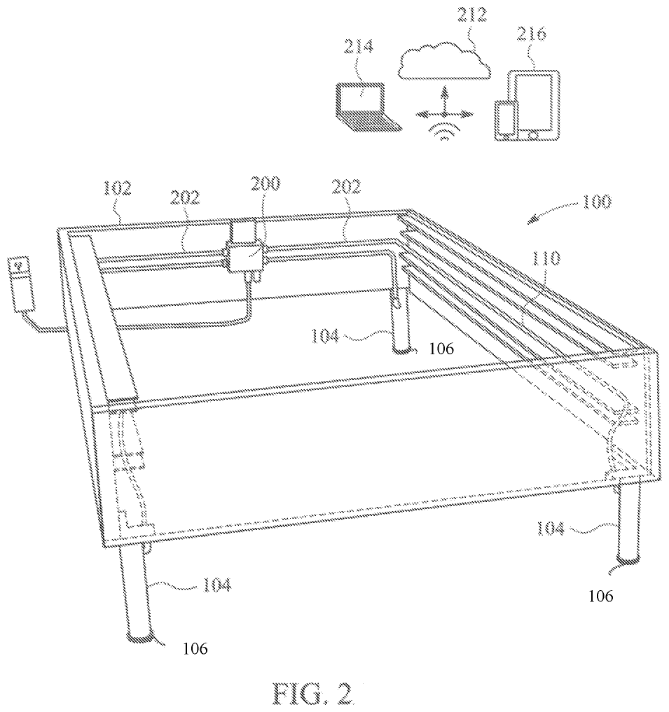

FIGS. 1 and 2 illustrate a system 100 for measuring data specific to a subject 10 using gravity. The system 100 can comprise a substrate 20 on which the subject 10 can lie. The substrate 20 is held in a frame 102 having multiple legs 104 extending from the frame 102 to a floor to support the substrate 20. Multiple load or other sensors 106 can be used, each load or other sensor 106 associated with a respective leg 104. Any point in which a load is transferred from the substrate 20 to the floor can have an intervening load or other sensor 106.

As illustrated in FIG. 2, a local controller 200 can be wired or wirelessly connected to the load or other sensors 106 and collects and processes the signals from the load or other sensors 106. The controller 200 can be attached to the frame 102 so that it is hidden from view, can be on the floor under the substrate or can be positioned anywhere a wireless transmission can be received from the load or other sensors 106 if transmission is wireless. Wiring 202 may electrically connect the load or other sensors 106 to the controller 200. The wiring 202 may be attached to an interior of the frame 102 and/or may be routed through the interior channels 110 of the frame 102. The controller 200 can collect and process signals from the load or other sensors 106. The controller 200 may also be configured to output power to the sensors and/or to printed circuit boards disposed in the load or other sensors 106.

The controller 200 can be programmed to control other devices based on the processed data, such as bedside or overhead lighting, door locks, electronic shades, fans, etc., the control of other devices also being wired or wireless. Alternatively, or in addition to, a cloud based computer 212 or off-site controller 214 can collect the signals directly from the load or other sensors 106 for processing or can collect raw or processed data from the controller 200. For example, the controller 200 may process the data in real time and control other local devices as disclosed herein, while the data is also sent to the off-site controller 214 that collects and stores the data over time. The controller 200 or the off-site controller 214 may transmit the processed data off-site for use by downstream third parties such a medical professionals, fitness trainers, family members, etc. The controller 200 or the off-site controller 214 can be tied to infrastructure that assists in collecting, analyzing, publishing, distributing, storing, machine learning, etc. Design of real-time data stream processing has been developed in an event-based form using an actor model of programming. This enables a producer/consumer model for algorithm components that provides a number of advantages over more traditional architectures. For example, it enables reuse and rapid prototyping of processing and algorithm modules. As another example, data streams can be enabled/disabled dynamically and routed to or from modules at any point within a group of modules comprising an algorithmic system, enabling computation to be location-independent (i.e., on a single device, combined with one or more additional devices or servers, on a server only, etc.).

The long-term collected data can be used in both a medical and home setting to learn and predict patterns of sleep, illness, etc. for a subject. As algorithms are continually developed, the long-term data can be reevaluated to learn more about the subject. Sleep patterns, weight gains and losses, changes in heart beat and respiration can together or individually indicate many different ailments. Alternatively, patterns of subjects who develop a particular ailment can be studied to see if there is a potential link between any of the specific patterns and the ailment.

The data can also be sent live from the controller 200 or the off-site controller 214 to a connected device 216, which can be wirelessly connected for wired. The connected device 216 can be, as examples, a mobile phone or home computer. Devices can subscribe to the signal, thereby becoming a connected device 216.

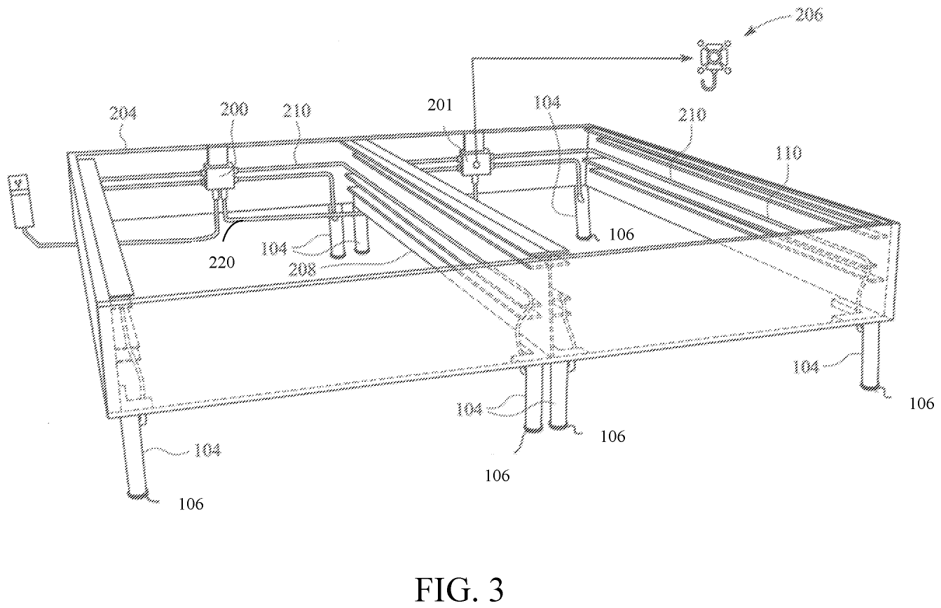

FIG. 3 is a top perspective view of a frame 204 for a bed 206 used with a substrate on which two or more subjects can lie. The bed 206 may include features similar to those of the bed 100 except as otherwise described. The bed 206 includes a frame 204 configured to support two or more subjects. The bed 206 may include eight legs, including one load or other sensor 106 disposed at each leg 104. In other embodiments, the bed may include nine legs 104 and nine load or other sensors 106, the additional sensor 106 disposed at the middle of the central frame member 208. In other embodiments, the bed 206 may include any arrangement of load or other sensors 106. Two controllers 200 and 201, for example, can be attached to the frame 204. The controllers 200 may be in wired or wireless communication with its respective sensors and optionally with each other. Each of the controllers 200 collects and processes signals from a subset of load or other sensors 106. For example, one controller 200 can collect and process signals from load or other sensors 106 (e.g. four load or other sensors) configured to support one subject lying on the bed 206. Another controller 200 can collect and process signals from the other load or other sensors 106 (e.g. four load or other sensors) configured to support the other subject lying on the bed 206. Wiring 210 may connect the load or other sensors 106 to either or both of the controllers 200 attached to the frame 204. In an implementation, wiring 220 can connect controllers 200 and 201. The wiring 210 may also connect the controllers 200. In other embodiments, the controllers may be in wireless communication with each other. In an implementation, one of the controllers 200 and 201, can process the signals collected by both of the controllers 200 and 201.

Examples of data determinations that can be made using the systems herein are described. The algorithms use the number of sensors and each sensor's angle and distance with respect to the other sensors. This information is predetermined. Software algorithms will automatically and continuously maintain a baseline weight calibration with the sensors so that any changes in weight due to changes in a mattress or bedding is accounted for.

The load or other sensors herein utilize macro signals and micro signals and process those signals to determine a variety of data, described herein. Macro signals are low frequency signals and are used to determine weight and center of mass, for example. The strength of the macro signal is directly influenced by the subject's proximity to each sensor.

Micro signals are also detected due to the heartbeat, respiration and to movement of blood throughout the body. Micro signals are higher frequency and can be more than 1000 times smaller than macro signals. The sensors detect the heart beating and can use its corresponding amplitude or phase data to determine where on the substrate the heart is located, thereby assisting in determining in what location, angular orientation, and body position the subject is laying as described and shown herein. In addition, the heart pumps blood in such a way that it causes top to bottom changes in weight. There is approximately seven pounds of blood in a human subject, and the movement of the blood causes small changes in weight that can be detected by the sensors. These directional changes are detected by the sensors. The strength of the signal is directly influenced by the subject's proximity to the sensor. Respiration is also detected by the sensors. Respiration will be a different amplitude and a different frequency than the heart beat and has different directional changes than those that occur with the flow of blood. Respiration can also be used to assist in determining the exact location, angular orientation, and body position of a subject on the substrate. These bio-signals of heart beat, respiration and directional movement of blood are used in combination with the macro signals to calculate a large amount of data about a subject, including the relative strength of the signal components from each of the sensors, enabling better isolation of a subject's bio-signal from noise and other subjects.

As a non-limiting example, the cardiac bio-signals in the torso area are out of phase with the signals in the leg regions. This allows the signals to be subtracted which almost eliminates common mode noise while allowing the bio-signals to be combined, increasing the signal to noise by as much as a factor of 3 db or 2× and lowering the common or external noise by a significant amount. By analyzing the phase differences in the 1 Hz to 10 Hz range (typically the heart beat range) the body position of a person laying on the bed can be determined. By analyzing the phase differences in the 0 to 0.5 Hz range, it can be determined if the person is supine, prone or laying on their side, as non-limiting examples.

Because signal strength is still quite small, the signal strength can be increased to a level more conducive to analysis by adding or subtracting signals, resulting in larger signals. The signals from each sensor can be combined by the signal from at least one, some, all or a combination of other sensors to increase the signal strength for higher resolution algorithmic analysis. The combining method can be linear or nonlinear addition, subtraction, multiplication or other transformations.

The controller can be programmed to cancel out external noise that is not associated with the subject laying on the bed. External noise, such as the beat of a bass or the vibrations caused by an air conditioner, register as the same type of signal on all load or other sensors and is therefore canceled out when deltas are combined during processing. Other noise cancellation techniques can be used including, but not limited to, subtraction, combination of the sensor data, adaptive filtering, wavelet transform, independent component analysis, principal component analysis, and/or other linear or nonlinear transforms.

Using superposition analysis, two subjects can be distinguished on one substrate. Superposition simplifies the analysis of the signal with multiple inputs. The usable signal equals the algebraic sum of the responses caused by each independent sensor acting alone. To ascertain the contribution of each individual source, all of the other sources must be calibrated first (turned off or set to zero). This procedure is followed for each source in turn, then the resultant responses are added to determine the true result. The resultant operation is the superposition of the various sources. By using signal strength and out-of-phase heart signal and/or respiration signal, individuals can be distinguished on the same substrate.

The controller can be programmed to provide dynamic center of mass location and movement vectors for the subject, while eliminating those from other subjects and inanimate objects or animals on the substrate. By leveraging multiple sensor assemblies that detect the z-axis of the force vector of gravity, and by discriminating and tracking the center of mass of multiple subjects as they enter and move on a substrate, not only can presence, motion and cardiac and respiratory signals for the subject be determined, but the signals of a single or multiple subjects on the substrate can be enhanced by applying the knowledge of location to the signal received. By analyzing the bio-signal's amplitude and phase in different frequency bands, the center of mass (location) for a subject can be obtained using multiple methods, examples of which include:
DC weight;
AC low band analysis of signal, center of mass (location), respiratory and body position identification of subject;
AC mid band analysis of signal center of mass and cardiac identification of subject; and
AC upper mid band identification of snorer or apnea events.

The data from the load or other sensor assemblies can be used to determine presence and location X and Y, angular orientation, and body positions of a subject on a substrate. Such information is useful for calculating in/out statistics for a subject such as: period of time spent in bed, time when subject fell asleep, time when subject woke up, time spent on back, time spent on side, period of time spent out of bed. The sensor assemblies can be in sleep mode until the presence of a subject is detected on the substrate, waking up the system.

Macro weight measurements can be used to measure the actual static weight of the subject as well as determine changes in weight over time. Weight loss or weight gain can be closely tracked as weight and changes in weight can be measured the entire time a subject is in bed every night. This information may be used to track how different activities or foods affect a person's weight. For example, excessive water retention could be tied to a particular food. In a medical setting, for example, a two-pound weight gain in one night or a five-pound weight gain in one week could raise an alarm that the patient is experiencing congestive heart failure. Unexplained weight loss or weight gain can indicate many medical conditions. The tracking of such unexplained change in weight can alert professionals that something is wrong.

Center of mass can be used to accurately heat and cool particular and limited space in a substrate such as a mattress, with the desired temperature tuned to the specific subject associated with the center of mass, without affecting other subjects on the substrate. Certain mattresses are known to provide heating and/or cooling. As non-limiting examples, a subject can set the controller to actuate the substrate to heat the portion of the substrate under the center of mass when the temperature of the room is below a certain temperature. The subject can set the controller to instruct the substrate to cool the portion of the substrate under the center of mass when the temperature of the room is above a certain temperature.

These macro weight measurements can also be used to determine a movement vector of the subject. Subject motion can be determined and recorded as a trend to determine amount and type of motion during a sleep session. This can determine a general restlessness level as well as other medical conditions such as “restless leg syndrome” or seizures.

Motion detection can also be used to report in real time a subject exiting from the substrate. Predictive bed exit is also possible as the position on the substrate as the subject moves is accurately detected, so movement toward the edge of a substrate is detected in real time. In a hospital or elder care setting, predictive bed exit can be used to prevent falls during bed exit, for example. An alarm might sound so that a staff member can assist the subject exit the substrate safely.

Data from the load or other sensors can be used to detect actual body positions of the subject on the substrate, such as whether the subject is on its back, side, or stomach. Data from the load or other sensors can be used to detect the angular orientation of the subject, whether the subject is aligned on the substrate vertically, horizontally, with his or her head at the foot of the substrate or head of the substrate, or at an angle across the substrate. The sensors can also detect changes in the body positions, or lack thereof. In a medical setting, this can be useful to determine if a subject should be turned to avoid bed sores. In a home or medical setting, firmness of the substrate can be adjusted based on the angular orientation and body position of the subject. For example, body position can be determined from the center of mass, position of heart beat and/or respiration, and directional changes due to blood flow.

Controlling external devices such as lights, ambient temperature, music players, televisions, alarms, coffee makers, door locks and shades can be tied to presence, motion and time, for example. As one example, the controller can collect signals from each load or other sensor, determine if the subject is asleep or awake and control at least one external device based on whether the subject is asleep or awake. The determination of whether a subject is asleep or awake is made based on changes in respiration, heart rate and frequency and/or force of movement. As another example, the controller can collect signals from each load or other sensor, determine that the subject previously on the substrate has exited the substrate and change a status of the at least one external device in response to the determination. As another example, the controller can collect signals from each load sensor, determine that the subject has laid down on the substrate and change a status of the at least one external device in response to the determination.

A light can be automatically dimmed or turned off by instructions from the controller to a controlled lighting device when presence on the substrate is detected. Electronic shades can be automatically closed when presence on the substrate is detected. A light can automatically be turned on when bed exit motion is detected or no presence is detected. A particular light, such as the light on a right side night stand, can be turned on when a subject on the right side of the substrate is detected as exiting the substrate on the right side. Electronic shades can be opened when motion indicating bed exit or no presence is detected. If a subject wants to wake up to natural light, shades can be programmed to open when movement is sensed indicating the subject has woken up. Sleep music can automatically be turned on when presence is detected on the substrate. Predetermined wait times can be programmed into the controller, such that the lights are not turned off or the sleep music is not started for ten minutes after presence is detected, as non-limiting examples.

The controller can be programmed to recognize patterns detected by the load or other sensors. The patterned signals may be in a certain frequency range that falls between the macro and the micro signals. For example, a subject may tap the substrate three times with his or her hand, creating a pattern. This pattern may indicate that the substrate would like the lights turned out. A pattern of four taps may indicate that the subject would like the shades closed, as non-limiting examples. Different patterns may result in different actions. The patterns may be associated with a location on the substrate. For example, three taps near the top right corner of the substrate can turn off lights while three taps near the base of the substrate may result in a portion of the substrate near the feet to be cooled. Patterns can be developed for medical facilities, in which a detected pattern may call a nurse.

While the figures illustrate the use of the load or other sensors with a bed as a substrate, it is contemplated that the load or other sensors can be used with couches, chairs, such as a desk chair, where a subject spends extended periods of time. A wheel chair can be equipped with the sensors to collect signals and provide valuable information about a patient. The sensors may be used in an automobile seat and may help to detect when a driver is falling asleep or his or her leg might go numb. Furthermore, the bed can be a baby's crib, a hospital bed, or any other kind of bed.

While the figures illustrate the use of the load sensors, other sensors, examples of which are described herein, can be used without departing from the scope of the specification or claims. Other sensors can be vibration sensors, pressure sensors, force sensors, motion sensors and accelerometers as non-limiting examples. In an implementation, the other sensors may be used instead of, in addition to or with the load sensors without departing from the scope of the specification or claims.

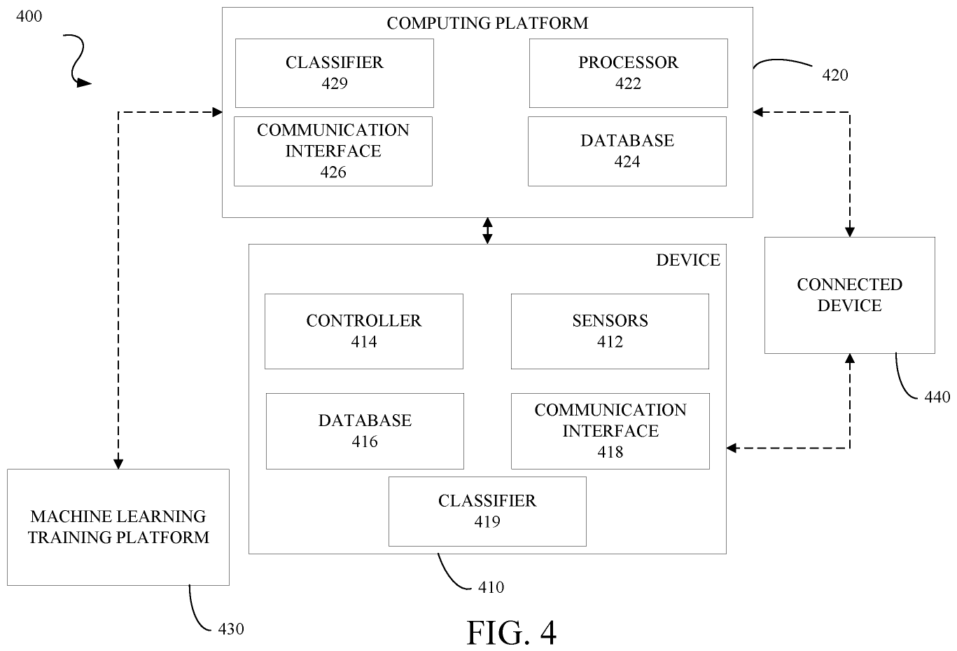

FIG. 4 is a system architecture for a multidimensional multivariate multiple sensor system (MMMSA) 400. The MMMSA 400 includes one or more devices 410 which are connected to or in communication with (collectively “connected to”) a computing platform 420. In an implementation, a machine learning training platform 430 may be connected to the computing platform 420. In an implementation, users may access the data via a connected device 440, which may receive data from the computing platform 420 or the device 410. The connections between the one or more devices 410, the computing platform 420, the machine learning training platform 430, and the connected device 440 can be wired, wireless, optical, combinations thereof and/or the like. The system architecture of the MMMSA 400 is illustrative and may include additional, fewer or different devices, entities and the like which may be similarly or differently architected without departing from the scope of the specification and claims herein. Moreover, the illustrated devices may perform other functions without departing from the scope of the specification and claims herein.

In an implementation, the device 410 can include one or more sensors 412, a controller 414, a database 416, and a communications interface 418. In an implementation, the device 410 can include a classifier 419 for applicable and appropriate machine learning techniques as described herein. The one or more sensors 412 can detect wave patterns of vibration, pressure, force, weight, presence, and motion due to subject(s) activity and/or configuration with respect to the one or more sensors 412. In an implementation, the one or more sensors 412 can generate more than one data stream. In an implementation, the one or sensors 412 can be the same type. In an implementation, the one or more sensors 412 can be time synchronized. In an implementation, the one or more sensors 412 can measure the partial force of gravity on substrate, furniture or other object. In an implementation, the one or more sensors 412 can independently capture multiple external sources of data in one stream (i.e. multivariate signal), for example, weight, heart rate, breathing rate, vibration, and motion from one or more subjects or objects. In an implementation, the data captured by each sensor 412 is correlated with the data captured by at least one, some, all or a combination of the other sensors 412. In an implementation, amplitude changes are correlated. In an implementation, rate and magnitude of changes are correlated. In an implementation, phase and direction of changes are correlated. In an implementation, the one or more sensors 412 placement triangulates the location of center of mass. In an implementation, the one or more sensors 412 can be placed under or built into the legs of a bed, chair, coach, etc. In an implementation, the one or more sensors 412 can be placed under or built into the edges of crib. In an implementation, the one or more sensors 412 can be placed under or built into the floor. In an implementation, the one or more sensors can be placed under or built into a surface area. In an implementation, the one or more sensors 412 locations are used to create a surface map that covers the entire area surrounded by sensors. In an implementation, the one or more sensors 412 can measure data from sources that are anywhere within the area surrounded by the sensors 412, which can be directly on top of the sensor 412, near the sensor 412, or distant from the sensor 412. The one or sensors 416 are not intrusive with respect to the subject(s).

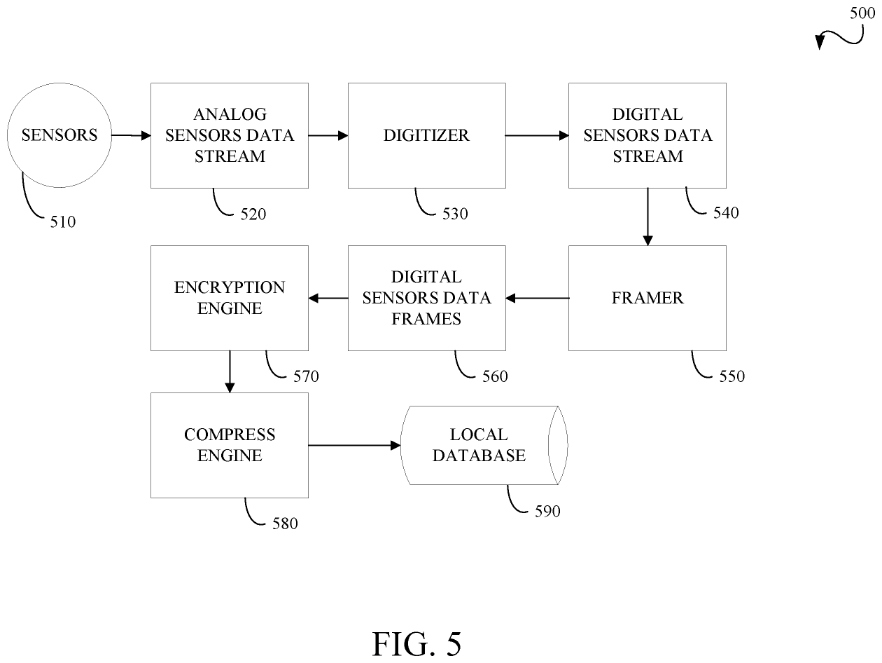

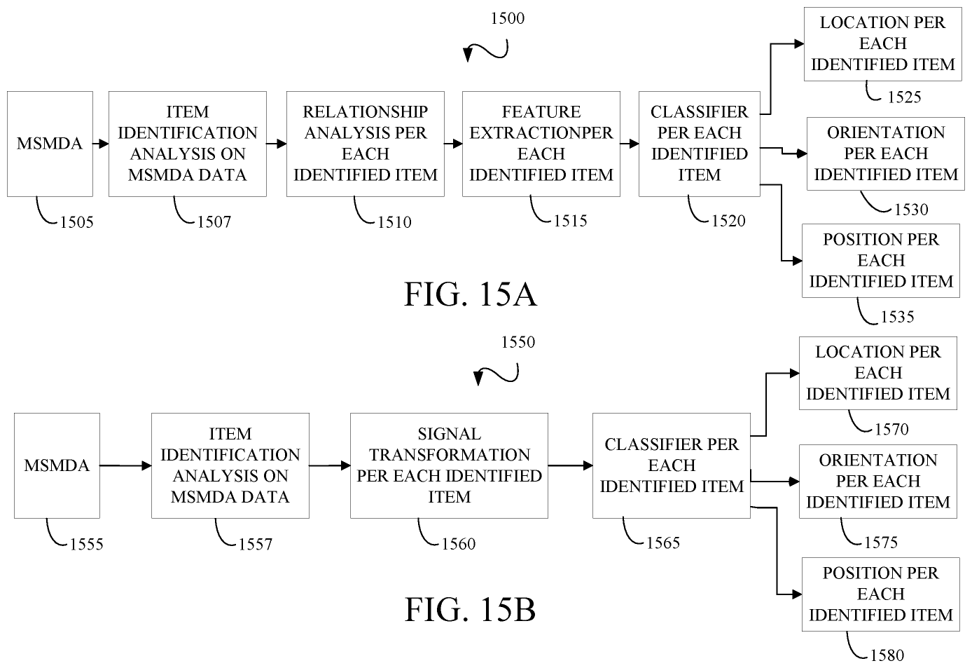

The controller 414 can apply the processes and algorithms described herein with respect to FIGS. 5-20 to the sensor data to determine biometric parameters and other person-specific information for single or multiple subjects at rest and in motion. The classifier 419 can apply the processes and algorithms described herein with respect to FIGS. 15A, 15B, 16, 19, and 20 to the sensor data to determine biometric parameters and other person-specific information for single or multiple subjects at rest and in motion. The classifier 419 can apply classifiers to the sensor data to determine the biometric parameters and other person-specific information via machine learning. In an implementation, the classifier 419 may be implemented by the controller 414. In an implementation, the sensor data and the biometric parameters and other person-specific information can be stored in the database 416. In an implementation, the sensor data, the biometric parameters and other person-specific information, and/or combinations thereof can be transmitted or sent via the communication interface 418 to the computing platform 420 for processing, storage, and/or combinations thereof. The communication interface 418 can be any interface and use any communications protocol to communicate or transfer data between origin and destination endpoints. In an implementation, the device 410 can be any platform or structure which uses the one or more sensors 412 to collect the data from a subject(s) for use by the controller 414 and/or computing platform 420 as described herein. For example, the device 410 may be a combination of the substrate 20, frame 102, legs 104, and multiple load or other sensors 106 as described in FIGS. 1-3. The device 410 and the elements therein may include other elements which may be desirable or necessary to implement the devices, systems, and methods described herein. However, because such elements and steps are well known in the art, and because they do not facilitate a better understanding of the disclosed embodiments, a discussion of such elements and steps may not be provided herein.

In an implementation, the computing platform 420 can include a processor 422, a database 424, and a communication interface 426. In an implementation, the computing platform 420 may include a classifier 429 for applicable and appropriate machine learning techniques as described herein. The processor 422 can obtain the sensor data from the sensors 412 or the controller 414 and can apply the processes and algorithms described herein with respect to FIGS. 5-20 to the sensor data to determine biometric parameters and other person-specific information for single or multiple subjects at rest and in motion. In an implementation, the processor 422 can obtain the biometric parameters and other person-specific information from the controller 414 to store in database 424 for temporal and other types of analysis. In an implementation, the classifier 429 can apply the processes and algorithms described herein with respect to FIGS. 15A, 15B, 16, 19, and 20 to the sensor data to determine biometric parameters and other person-specific information for single or multiple subjects at rest and in motion. The classifier 429 can apply classifiers to the sensor data to determine the biometric parameters and other person-specific information via machine learning. In an implementation, the classifier 429 may be implemented by the processor 422. In an implementation, the sensor data and the biometric parameters and other person-specific information can be stored in the database 424. The communication interface 426 can be any interface and use any communications protocol to communicate or transfer data between origin and destination endpoints. In an implementation, the computing platform 420 may be a cloud-based platform. In an implementation, the processor 422 can be the cloud-based computer 212 or off-site controller 214. The computing platform 420 and elements therein may include other elements which may be desirable or necessary to implement the devices, systems, and methods described herein. However, because such elements and steps are well known in the art, and because they do not facilitate a better understanding of the disclosed embodiments, a discussion of such elements and steps may not be provided herein.

In an implementation, the machine learning training platform 430 can access and process sensor data to train and generate classifiers. The classifiers can be transmitted or sent to the classifier 429 or to the classifier 419.

FIG. 5 is a processing pipeline 500 for obtaining sensor data such as, but not limited to, load sensor data and other sensor data. An analog sensors data stream 520 is received from the sensors 510. A digitizer 530 digitizes the analog sensors data stream into a digital sensors data stream 540. A framer 550 generates digital sensors data frames 560 from the digital sensors data stream 540 which includes all the digital sensors data stream values within a fixed or adaptive time window. An encryption engine 570 encodes the digital sensors data frames 560 such that the data is protected from unauthorized access. A compression engine 580 compresses the encrypted data to reduce the size of the data that is going to be saved in the database 590. This reduces cost and provides faster access during read time. The processing pipeline 500 shown in FIG. 5 is illustrative and can include any, all, none or a combination of the blocks or modules shown in FIG. 5. The processing order shown in FIG. 5 is illustrative and the processing order may vary without departing from the scope of the specification or claims.

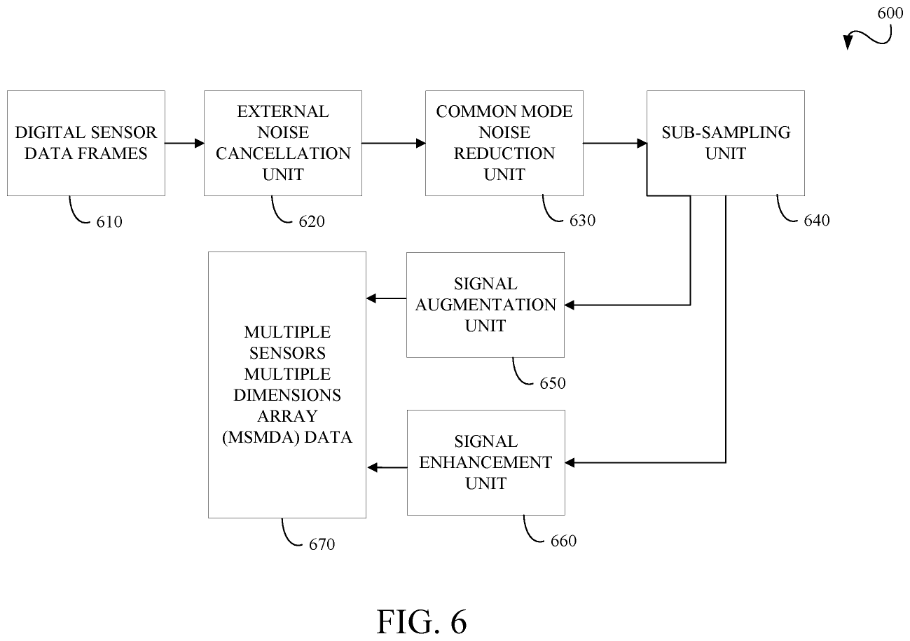

FIG. 6 is a pre-processing pipeline 600 for processing the sensor data into multiple sensors multiple dimensions array (MSMDA) data. The pre-processing pipeline 600 shown in FIG. 6 is illustrative and can include any, all, none or a combination of the blocks or modules shown in FIG. 6. The processing order shown in FIG. 6 is illustrative and the processing order may vary without departing from the scope of the specification or claims. The pre-processing pipeline 600 processes digital sensor data frames 610. An external noise cancellation unit 620 removes or attenuates noise sources that might have the same or different level of impact on each sensor. The external noise cancellation unit 620 can use a variety of techniques including, but not limited to, subtraction, combination of the input data frames, adaptive filtering, wavelet transform, independent component analysis, principal component analysis, and/or other linear or nonlinear transforms. A common mode noise reduction unit 630 removes or attenuates noises which are captured equally by all sensors. The common mode noise reduction unit 630 may use a variety of techniques including, but not limited to, subtraction, combination of the input data frames, adaptive filtering, wavelet transform, independent component analysis, principal component analysis, and/or other linear or nonlinear transforms. A subsampling unit 640 samples the digital sensor data and can include downsampling, upsampling or resampling. The subsampling unit 640 can be implemented as a multi-stage sampling or multi-phase sampling. A signal augmentation unit 650 can improve the energy of the data or content. The signal augmentation unit 650 can be implemented as scaling, normalization, log transformation, power transformation, linear or nonlinear combination of input data frames and/or other transformations on the input data frames. A signal enhancement unit 660 can improve the signal to noise ratio of the input data. The signal enhancement unit 660 can be implemented as a linear or nonlinear combination of input data frames. For example, the signal enhancement unit 660 may combine the signal deltas to increase the signal strength for higher resolution algorithmic analysis. The pre-processing pipeline 600 outputs MSMDA data 670, which is the primary input to the methods described herein.

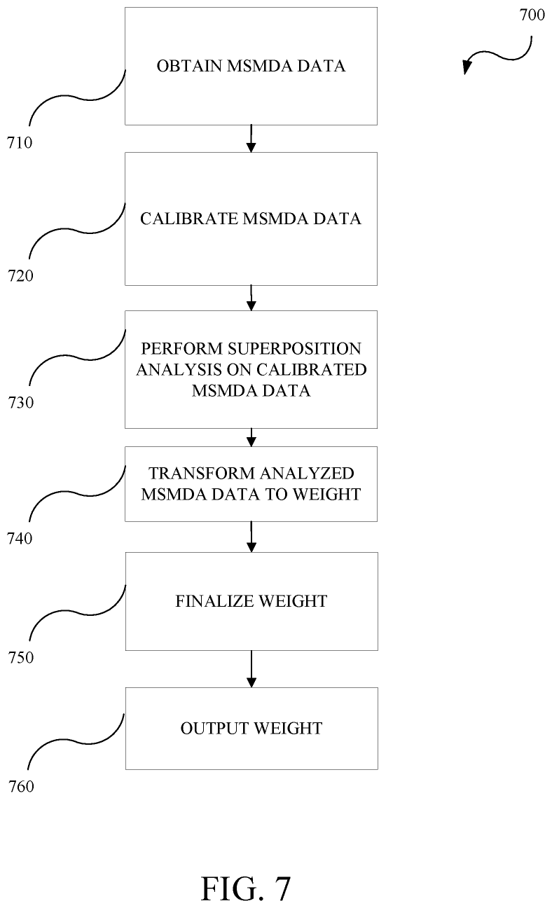

FIG. 7 is a flowchart of a method 700 for determining weight from the MSMDA data. The method 700 includes: obtaining 710 the MSMDA data; calibrating 720 the MSMDA data; performing 730 superposition analysis on the calibrated MSMDA data; transforming 740 the MSMDA data to weight; finalizing 750 the weight; and outputting 760 the weight.

The method 700 includes obtaining 710 the MSMDA data. The MSMDA data is generated from the pre-processing pipeline 600 as described.

The method 700 includes calibrating 720 the MSMDA data. The calibration process compares the multiple sensors readings against an expected value or range. If the values are different, the MSMDA data is adjusted to calibrate to the expected value range. Calibration is implemented by turning off all other sources (i.e. set them to zero) in order to determine the weight of the new object. For example, the weight of the bed, bedding and pillow are determined prior to the new object. A baseline is established of the device, for example, prior to use. In an implementation, once a subject or object (collectively “item”) is on the device, an item baseline is determined and saved. This is done so that data from a device having multiple items can be correctly processed using the methods described herein.

The method 700 includes performing 730 superposition analysis on the calibrated MSMDA data. Superposition analysis provides the sum of the readings caused by each independent sensor acting alone. The superposition analysis can be implemented as an algebraic sum, a weighted sum, or a nonlinear sum of the responses from all the sensors.

The method 700 includes transforming 740 the MSMDA data to weight. A variety of known or to be known techniques can be used to transform the sensor data, i.e. the MSMDA data, to weight.

The method 700 includes finalizing 750 the weight. In an implementation, finalizing the weight can include smoothing, checking against a range, checking against a dictionary, or a past value. In an implementation, finalizing the weight can include adjustments due to other factors such as bed type, bed size, location of the sleeper, position of the sleeper, orientation of the sleeper, and the like.

The method 700 includes and outputting 760 the weight. The weight is stored for use in the methods described herein.

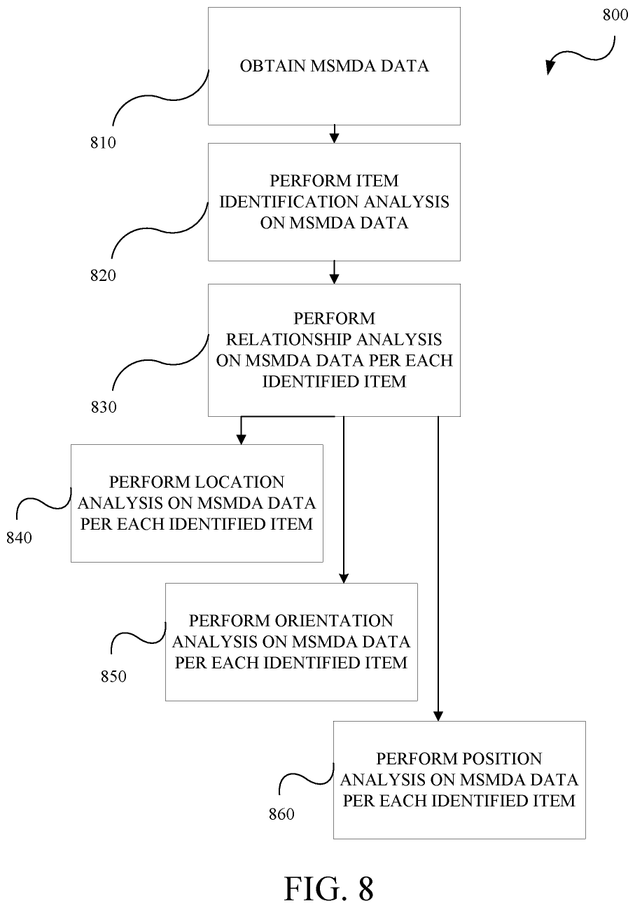

FIG. 8 is a flowchart of a method 800 for performing spatial analysis using the MSMDA data. The method 800 includes: obtaining 810 the MSMDA data; performing subject and/or object (collectively “item”) identification analysis on MSMDA data; performing 830 relationship analysis on the MSMDA data; performing 840 location analysis on the MSMDA data; performing 850 angular orientation analysis on the MSMDA data; and performing 860 body position analysis on the MSMDA data.

The method 800 includes obtaining 810 the MSMDA data. The MSMDA data is generated from the pre-processing pipeline 600 as described.

The method 800 includes performing 820 subject and/or object (collectively “item”) identification analysis on MSMDA data. The item identification determines the number of items on the surface area of the substrate, for example, and the order they got on the surface area. For example, the method determines when a first sleeper gets in bed, when a second sleeper gets in bed, when either sleepers gets out of the bed (it could be that the first sleeper gets out first or the second sleeper gets out first). The method can determine if an object has been placed on the bed. The method can further determine if an animal has jumped on the bed or a kid has got in bed. The method assigns a label to each item to track the sequence of bed entry and exit for each item. The method can use the calibration 720 of FIG. 7 to perform item identification. In an implementation, other techniques can be used, such as but not limited to, independent component analysis, multiple threshold analysis and pattern matching analysis to identify multiple items.

The method 800 includes performing 830 relationship analysis on the MSMDA data. For each identified item, the relationship analysis identifies individual sensors or combination of sensors which are correlated, associated, dependent, or otherwise related based on some parameter or function. This includes finding linear and nonlinear relationships between any two or more combinations using correlation, dependence and association analysis. For example, the relationship can be defined in terms of amplitude, rate of changes, magnitude of changes, phase changes, direction of changes, and/or combinations thereof.

The method 800 includes performing 840 location analysis on the MSMDA data. For each identified item, the location analysis determines where a subject/object is sleeping or placed, for example, on a bed. For example, the subject can be sleeping at a right edge, center, top, corner, or an x-y coordinate.

The method 800 includes performing 850 angular orientation analysis on the MSMDA data. For each item, the orientation analysis determines what angle subject/object is sleeping or placed, for example, on a bed. For example, the subject can be sleeping vertically, diagonally, horizontally and the like.

The method 800 includes performing 860 body position analysis on the MSMDA data. For each identified subject, the body position analysis determines how a subject is sleeping, for example, on a bed. For example, the subject can be sleeping in a fetal position, on the back, supine, on the right side, prone, and the like.

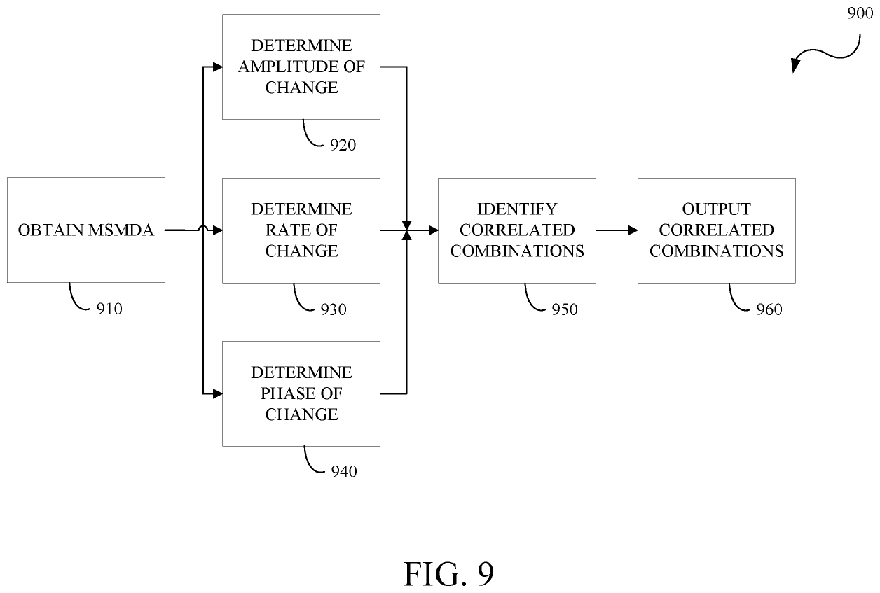

FIG. 9 is a flowchart of a method 900 for performing a relationship analysis using the MSMDA data for each identified item. The method 900 includes: obtaining 910 the MSMDA data; determining 920 amplitude of change; determining 930 rate of change; determining 940 phase of change; identifying 950 correlated combinations; and outputting 960 the correlated combinations.

The method 900 includes obtaining 910 the MSMDA data. The MSMDA data is generated from the pre-processing pipeline 600 as described.

The method 900 includes determining 920 amplitude of change, determining 930 rate of change, and determining 940 phase of change. For a given pair or combination of sensors, these processes identify the amplitude of change, rate of change, and phase of change by applying time domain, spectral domain, and time-frequency techniques.

The method 900 includes identifying 950 correlated combinations. All combinations are sorted based on a metric such as, but not limited to, correlation coefficients. The first N combinations with the highest value of a correlation metric are selected. For each selected combination, a “1” is assigned to any other combination which has a similar change in amplitude, rate or phase, a “−1” is assigned to any other combination which has an opposite amplitude rate or phase change, and a “0” is assigned otherwise, where same or opposite is determined by the value of the correlation metric. A positive correlation coefficient indicates same directional change and a negative correlation coefficient indicates an inverse directional relation. For example, a phase between 0 and 180 indicates same angular change, and a phase between −180 and 0 shows opposite angular relation. For example, the sign of the differential rate change if positive shows changes in the same direction and if negative shows changes in the opposite direction.

The method 900 includes outputting 960 the correlated combinations. The assigned correlated combinations are output for use in the methods described herein.

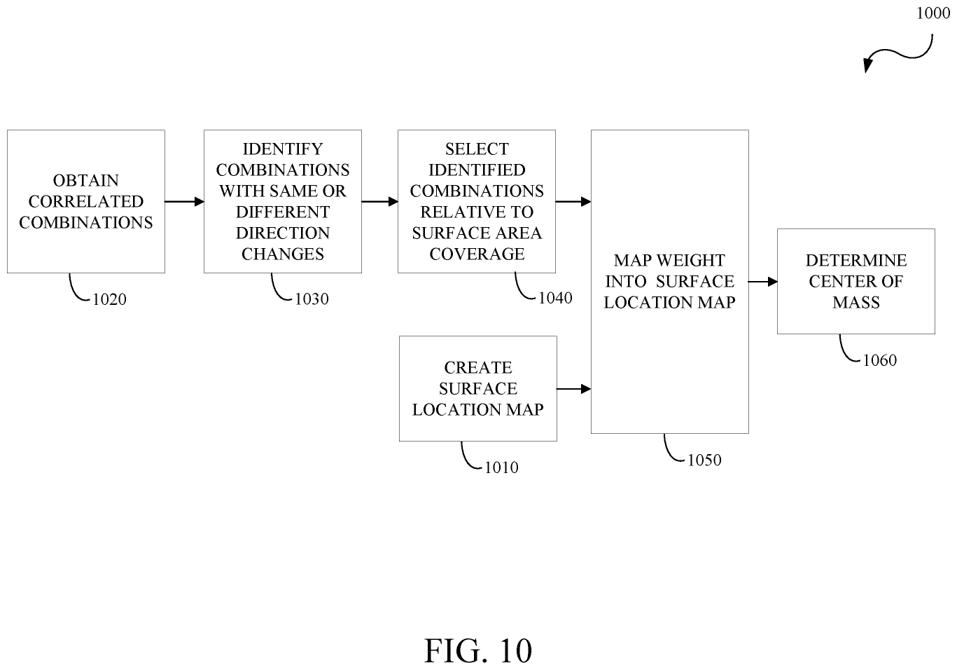

FIG. 10 is a flowchart for a method 1000 for performing location analysis using the MSMDA data for each identified item. The method 1000 includes creating 1010 a surface location map; obtaining 1020 correlated combinations; identifying 1030 combinations with same or different direction changes; selecting 1040 identified correlated combinations relative to the surface area coverage; mapping 1050 weight into surface location map; and determining 1060 location or center of mass.

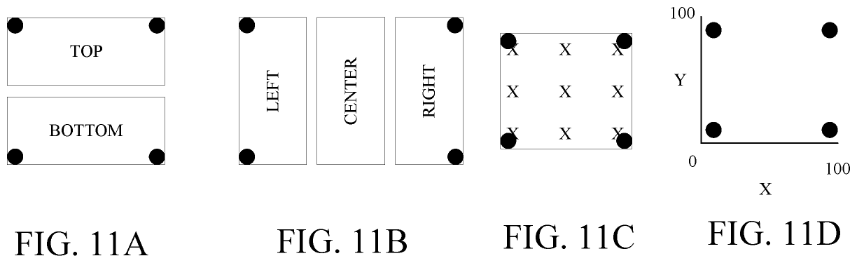

The method 1000 includes creating 1010 a surface location map. A two-dimensional surface location map is generated to represent the surface of a substrate, furniture or other object. FIGS. 11A-D show example surface location maps for a multidimensional multivariate multiple sensors system with 4 sensors. FIG. 11A shows mapping the surface into a top section and bottom section. FIG. 11B shows mapping the surface into left, center, and right sections. FIG. 11C shows mapping the surface into 9 coordinates: top left, middle top, top right, middle right, bottom right, middle bottom, bottom left, middle left, and center. FIG. 11D shows mapping the surface into a two dimensional X-Y coordinate, where X and Y are in the range of 0-100 such that (X,Y)=(0,0) represents the bottom left corner of the surface, (X,Y)=(100,100) represent the top right corner of the surface, and (X,Y)=(50,50) shows the center of the surface. The coordinate system is illustrative and other formats can be used. The surface location maps are illustrative and other formats can be used.

The method 1000 includes obtaining 1020 correlated combinations. The correlated combinations data is obtained from the relationship analysis method 900 of FIG. 9.

The method 1000 includes identifying 1030 combinations with same or different direction changes. The assignment values of the correlated combinations are reviewed to identify which combinations have the same or different direction changes.

The method 1000 includes selecting 1040 identified correlated combinations relative to the surface coverage area. The directionally correlated combinations are down selected to those which represent the surface coverage area surrounded by the sensors. The term “surface coverage area” refers to the area defined by the sensor placement. For example, the surface of a bed, a coach, floor surface, etc. For example, each item may have a different surface coverage area depending on placement on the substrate, for example.

The method 1000 includes mapping 1050 weight into a surface location map and determining 1060 the location or center of mass. The correlated combinations representing the surface and the surface location map are used to map the center of mass (i.e. weight). For example, in a top vs. bottom mapping, if the combination of top sensors are correlated and directionally change in the same direction, and the combination of bottom sensors are correlated and change in the same direction, and top and bottom combinations have opposite direction changes, where top shows an increase and bottom shows a decrease or vice versa, the center of mass is determined to be at the top section. In an implementation, any of the two-dimensional surface location maps can be used to determine the location or center of mass. In an implementation, the surface location map is selected based on level of resolution needed for analysis.

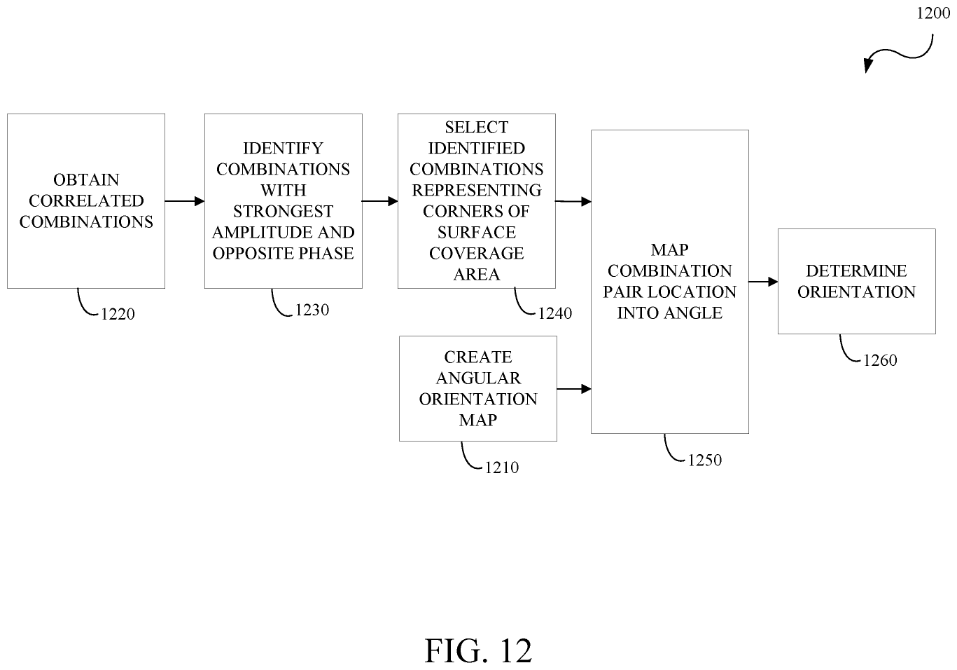

FIG. 12 is a flowchart of a method 1200 for performing angular orientation analysis using the MSMDA data for each item. The method 1200 includes: creating 1210 an angular orientation map; obtaining 1220 correlated combinations; identifying 1230 combinations with strongest amplitude and opposite phase; selecting 1240 identified correlated combinations representing boundaries of the surface coverage area; mapping 1250 combination pair location into angle using the orientation map; and determining 1060 angular orientation.

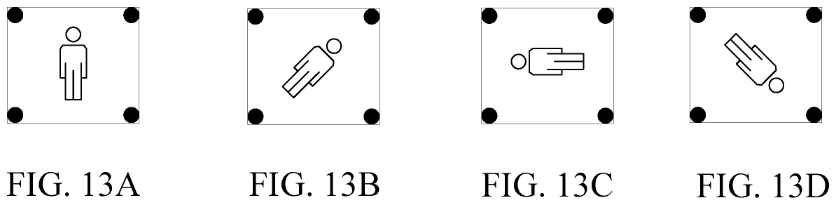

The method 1200 includes creating 1210 an angular orientation map. Angular orientation maps are created to represent the subject/sleeper/user on the substrate, furniture or other object. FIGS. 13A-D illustrate different angular orientation maps. FIG. 13A shows a vertical orientation map. FIG. 13B shows a diagonal orientation map. FIG. 13C shows a horizontal orientation map. FIG. 13D shows a reverse diagonal orientation map. The angular orientation maps are illustrative and other formats can be used.

The method 1200 includes obtaining 1220 correlated combinations. The correlated combinations data is obtained from the relationship analysis method 900 of FIG. 9.

The method 1200 includes identifying 1230 combinations with strongest amplitude and opposite phase. The correlated combinations are reviewed to determine the correlated combinations which have the strongest amplitude and opposite phase.

The method 1200 includes selecting 1240 identified correlated combinations representing boundaries of the surface coverage area. The identified correlated combinations are down selected to those which represent the boundaries of the surface coverage area surrounded by the sensors.

The method 1200 includes mapping 1250 combination pair location into angle using the orientation map and determining 1060 the orientation. A combination pair location refers to the coordinates of the individual sensors that form the combination. The selected correlated combinations (the combination pair location) are mapped into an angle using the orientation map. For example, in a vertical mapping, if the combination of top sensors have the strongest amplitude, and the combination of top sensors have the opposite phase to the combination of bottom sensors, orientation is determined to be vertical. In an illustrative example of a combination pair location, imagine the combination of TOP RIGHT-BOTTOM LEFT sensors has the strongest amplitude and opposite phase. Then, the location of the two sensors that have formed this combination (TOP RIGHT and BOTTOM LEFT) will be mapped into an angle (for example, 45 degrees referenced to the lower right corner of the bed), which indicates a diagonal orientation.

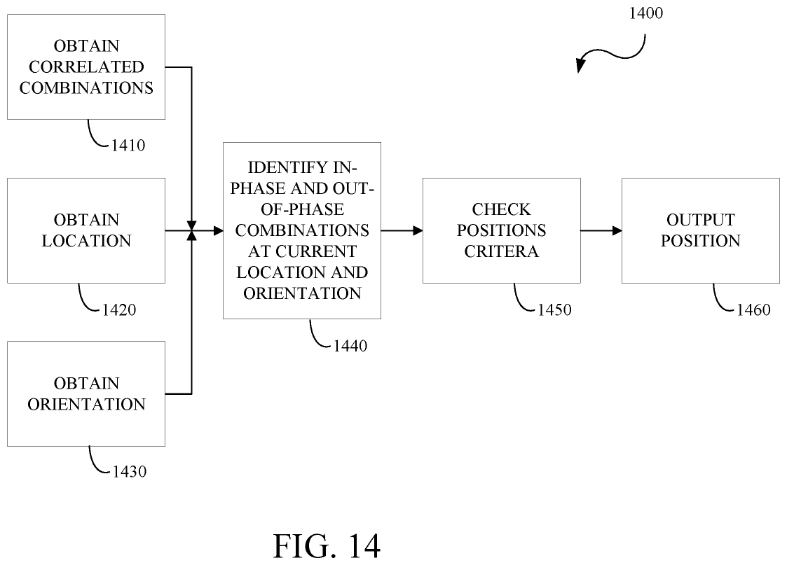

FIG. 14 is a flowchart of a method 1400 for performing body position analysis using the MSMDA data for each item. The method 1400 includes: obtaining 1410 correlated combinations; obtaining 1420 location data; obtaining 1430 angular orientation data; identifying 1440 in-phase and out-of-phase combinations at current location and angular orientation; checking 1450 body position data; and outputting 1460 body position.

The method 1400 includes obtaining 1410 correlated combinations data, obtaining 1420 location data, and obtaining 1430 angular orientation data. The correlated combinations data is obtained from the relationship analysis method 900 of FIG. 9, the location data is obtained from the location analysis method 1000 of FIG. 10, and the angular orientation data is obtained from the angular orientation analysis method 1200 of FIG. 12.

The method 1400 includes identifying 1440 in-phase and out-of-phase combinations at the current location and angular orientation. The location and angular orientation data sets are used to define in-phase and out-of-phase relations relative to the item's current location and angular orientation, which will help limiting the data to be analyzed. Directional changes can be the same or different. In-phase refers to a pair of combinations that have the same directional change and out-of-phase refers to a pair of combinations that have different directional changes.

The method 1400 includes checking 1450 body positions criteria. The body positions can be, for example, supine, left side, right side, prone, and the like. The in-phase and out-of-phase determinations are used to determine the body position. In an implementation, a lookup table can be used to determine the body position. In an example, a look-up table can use an in-phase and out-of-phase combinations index to look for the corresponding body position. For example, anytime the combination of sensors 1 and 4 are in-phase and the combination of sensor 2 and 4 are out-of-phase, body position is supine. In an implementation, the in-phase and out-of-phase determinations in time domain, spectral domain, or time-frequency domain are matched against conditions for a given body position. In an implementation, a classifier can be used that is trained to determine the body position.

The method 1400 includes outputting 1460 position. The determined body position can be saved for methods described herein.

FIGS. 15A-B are block diagrams for performing spatial analysis using supervised and unsupervised machine learning, respectively. Machine learning techniques can be used to do spatial analysis. A classifier or a set of classifiers can be trained to learn and determine location, angular orientation, and body position. If a set of classifiers are used, each of the location, angular orientation, and body position determinations would have a separate classifier, respectively. Classification can be implemented as supervised classifiers or unsupervised classifiers.

FIG. 15A is a block diagram 1500 for performing spatial analysis using a supervised classifier. MSMDA data is obtained 1505 as training or inference data from the output of the pre-processing pipeline 600 of FIG. 6. A relationship analysis is performed 1510 on the MSMDA data to generate a feature set 1515 and mapped to a kernel space. An item identification analysis is performed 1507 on the MSMDA data. The item identification determines the number of items on the surface area, and the order they got on the surface area. For example, it determines when a first sleeper gets in bed, when a second sleeper gets in bed, when either sleepers gets out of the bed (it could be that the first sleeper gets out first or the second sleeper gets out first). The analysis can determine if an item has been placed on the bed. The analysis can further determine if an animal has jumped on the bed or a kid has got in bed. The analysis assigns a label to each item to track the sequence of bed entry and exit for each item. The analysis can use the calibration 720 of FIG. 7 to perform item identification. Other techniques such as independent component analysis, multiple threshold analysis and pattern matching analysis to identify multiple items can be used. A classifier 1520 is trained on the feature set 1515 so that the classifier 1520 is able to classify unseen data. Once trained, the classifier 1520 can use specific classifiers to determine location 1525, orientation 1530, and position 1535. The supervised training requires providing a set of labels (i.e. annotations) for the training data. The labels can be provided by human or programmatically using an algorithm that pre-annotates the input data. For example, this training can be done using the machine learning training platform 430. In an implementation, a device 410 can do the training. The classifier 1520 can be a machine learning classifier or a deep learning classifier.

FIG. 15B is a block diagram 1550 for performing spatial analysis using an unsupervised classifier. In the unsupervised approach, the training may be performed only with the analysis of the input data only and not requiring annotations. This can include unsupervised clustering of the data using any of the following methods: k-means clustering, hierarchical clustering, mixture models, self-organizing maps, hidden Markov models, a deep convolutional neural network (CNN), a recursive network, or a long short-term memory (LSTM) network. In an implementation, an item identification analysis is performed 1557 on the MSMDA data 1555. The item identification determines the number of items on the surface area, and the order they got on the surface area. For example, it determines when a first sleeper gets in bed, when a second sleeper gets in bed, when either sleepers gets out of the bed (it could be that the first sleeper gets out first or the second sleeper gets out first). The analysis can determine if an item has been placed on the bed. The analysis can further determine if an animal has jumped on the bed or a kid has got in bed. The analysis assigns a label to each item to track the sequence of bed entry and exit for each item. The analysis can use the calibration 720 of FIG. 7 to perform item identification. Other techniques such as independent component analysis, multiple threshold analysis and pattern matching analysis to identify multiple items can be used. An unsupervised classifier 1565, on a per identified item basis, may use the MSMDA data 1555 or apply a signal transformation 1560 to the MSMDA data 1555 to transform the input data into a space that is more suitable for classification. For example, the signal transformation 1560 can be, but is not limited to, wavelet, cosine, fast Fourier transform (FFT), short time FFT, and the like. The unsupervised classifier 1565 can then apply the specific classifiers to determine location 1570, orientation 1575, and position 1580. The unsupervised classifier 1565 can be a machine learning classifier or a deep learning classifier. The unsupervised classifier 1565 can be an unsupervised classifier or a set of unsupervised classifiers for location, angular orientation, and body position classification.

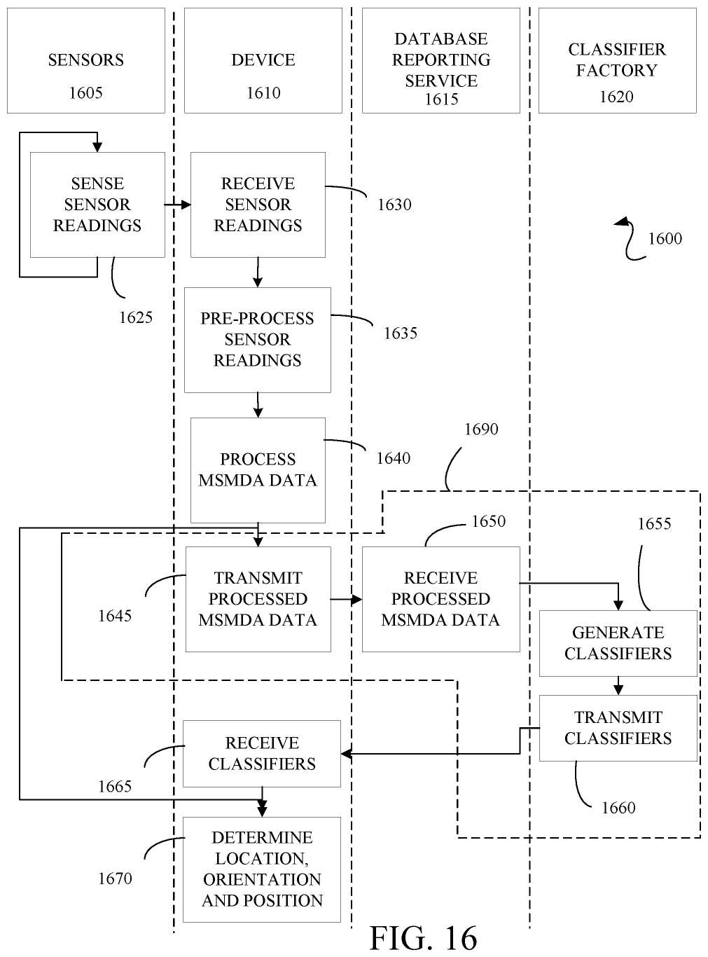

FIG. 16 is a swim lane diagram 1600 for performing location, orientation and position analysis for each item using machine learning. The swim lane diagram 1600 includes sensors 1605, device(s) 1610, database reporting service 1615, and a classifier factory 1620. In an implementation, the database reporting service 1615 and the classifier factory 1620 can be implemented at computing platform 420, for example. In an implementation, the database reporting service 1615 and the classifier factory 1620 can be implemented at the device 1610 or device 410 in FIG. 4, for example.

At training time, sensor readings 1625 from the sensors 1605 are received by the device 1610 (1630). The sensor data is pre-processed 1635 to form MSMDA data and then the MSMDA data is processed 1640 (for example, to generate features or to map into the kernel space) as described herein. The device 1610 transmits the processed MSMDA data 1645 to the database reporting service 1615. The database reporting service 1615 receives the processed MSMDA data 1650. The classifier factory 1620 generates classifiers 1655 from the received the processed MSMDA data, and annotations (if supervised). The classifier factory 1620 transmits the classifiers 1660 to the device(s) 1610. The device(s) 1610 receive the classifiers 1665 and use the classifiers to determine location, angular orientation and body position 1670 per each identified item. In this instance, transmitting the processed MSMDA data 1645, receiving the processed sensor readings 1650, generating classifiers 1655, and transmitting the classifiers 1660 are performed during training time as training blocks 1690.

At operation time, the sensor readings 1625 from the sensors 1605 are received by the device 1610 (1630). The sensor readings are pre-processed 1635 to form MSMDA data and then the MSMDA data is processed 1640 (for example, to generate features or to map into the kernel space) as described herein and fed into the classifier to determine and classify location, angular orientation, and body position 1670.

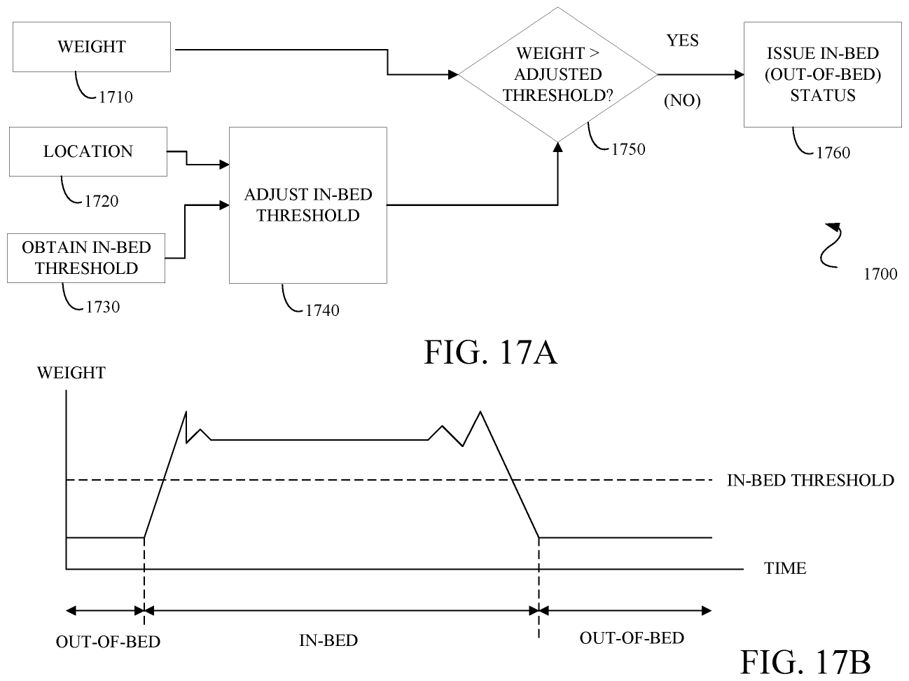

FIGS. 17A-B is a flowchart for a method 1700 for detecting bed presence for each item and an example graphical representation. The method 1700 includes: obtaining 1710 weight data; obtaining 1720 location data; obtaining 1730 an in-bed threshold; adjusting 1740 the in-bed threshold; determining 1750 if the weight is greater than the adjusted threshold; and issuing 1760 a status alert. FIG. 17B shows a graphical representation of the weight versus in-bed threshold determination with respect to in-bed and out-of-bed.

The method 1700 includes obtaining 1710 weight data and obtaining 1720 location data. The weight data is obtained from the method 700 and the location data is obtained using the multiple methods described herein.

The method 1700 includes obtaining 1730 an in-bed threshold. The in-bed threshold can be pre-defined and set in the system. The in-bed threshold can be obtained from a look-up table where different thresholds are used for different sensor types, different bed types, different sleeper ages and the like.

The method 1700 includes adjusting 1740 the in-bed threshold. The in-bed threshold can be adjusted based on the location data. For example, the threshold when the subject is sitting on the edge of the bed might be different than the threshold when the subject is laying.

The method 1700 includes determining 1750 if the weight is greater than the adjusted threshold. The weight is checked against the adjusted threshold.

The method 1700 includes issuing 1760 a status alert. If the weight is greater than the adjusted threshold then an in-bed status is shown or sent. If the weight is not greater than the adjusted threshold then an out-of-bed status is shown or sent. In an implementation, the status can be used to alert personnel and the like.

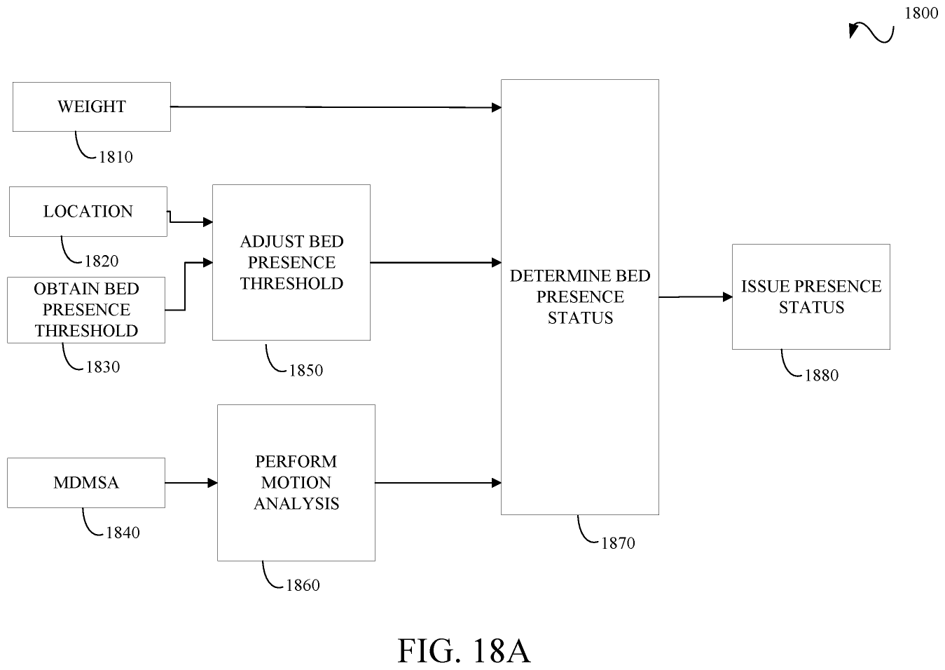

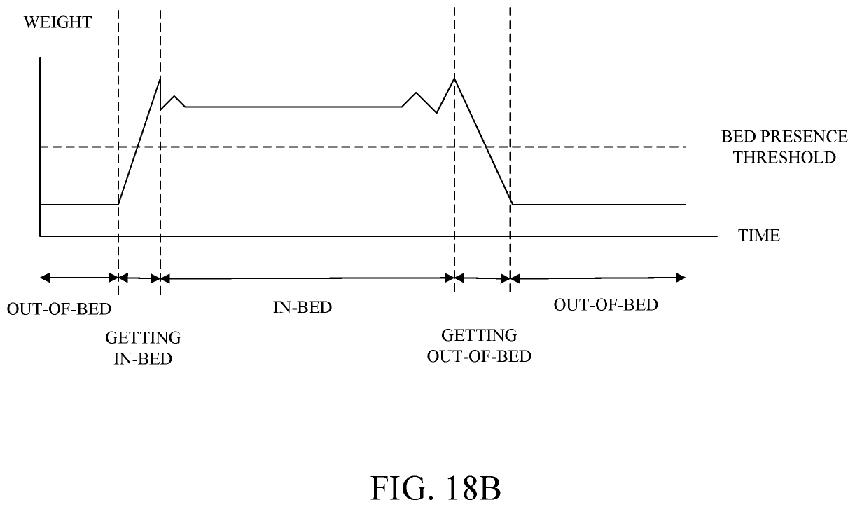

FIGS. 18A-B is a flowchart for a method 1800 detecting bed presence for each item with in/out transitions and an example graphical representation. The method 1800 includes: obtaining 1810 weight data; obtaining 1820 location data; obtaining 1830 a bed presence threshold; obtaining 1840 MDMSA data; adjusting 1850 the bed presence threshold; performing 1860 motion analysis using the MDMSA data; determining 1870 bed presence status; and issuing 1880 a status alert. FIG. 18B shows a graphical representation of the weight versus bed presence threshold determination with respect to in-bed, getting in bed, getting out of bed and out-of-bed.

The method 1800 includes obtaining 1810 weight data, obtaining 1820 location data, and obtaining 1840 MDMSA data. The weight data is obtained from the method 700 and the location data is obtained using the multiple methods described herein. The MDMSA data is obtained from the method 600 as described herein.

The method 1800 includes obtaining 1840 a bed presence threshold. The bed presence threshold can be pre-defined and set in the system. The bed presence threshold can be obtained from a lookup table where different thresholds are used for different sensor types, different bed types, different sleeper ages and the like. In an implementation, the bed presence threshold can include multiple thresholds such as in-bed vs. out-of-bed threshold and a getting in-bed vs getting out-of-bed threshold.

The method 1800 includes adjusting 1850 the bed presence threshold. The bed presence threshold can be adjusted based on the location data. For example, the bed presence threshold when the subject is sitting on the edge of the bed might be different than the bed presence threshold when the subject is laying.

The method 1800 includes performing 1860 motion analysis using the MDMSA data. Motion analysis is performed using the MDMSA data to determine if the subject is moving, how much the subject is moving, at what speed the subject is moving, and for how long the subject has been moving.

The method 1800 includes determining 1870 bed presence status. The bed presence status can be determined from the weight, the adjusted bed presence threshold, and the motion analysis determination using a variety of techniques. In an implementation, a look up table can be used. An example lookup table can use weight, motion and adjusted bed presence threshold to look for the corresponding bed presence status. For example, the look up table may include a column corresponding to weight values or ranges, a column for motion values, levels, or ranges, and a column for presence status. In an implementation, pattern matching can be used. In an implementation, threshold analysis can be used.

The method 1800 includes issuing 1880 a status alert. In an implementation, the status can be used to alert personnel and the like if the subject is moving with respect to a previous status on the device. In an implementation, the status can be used to activate light and/or other devices to assist the subject.

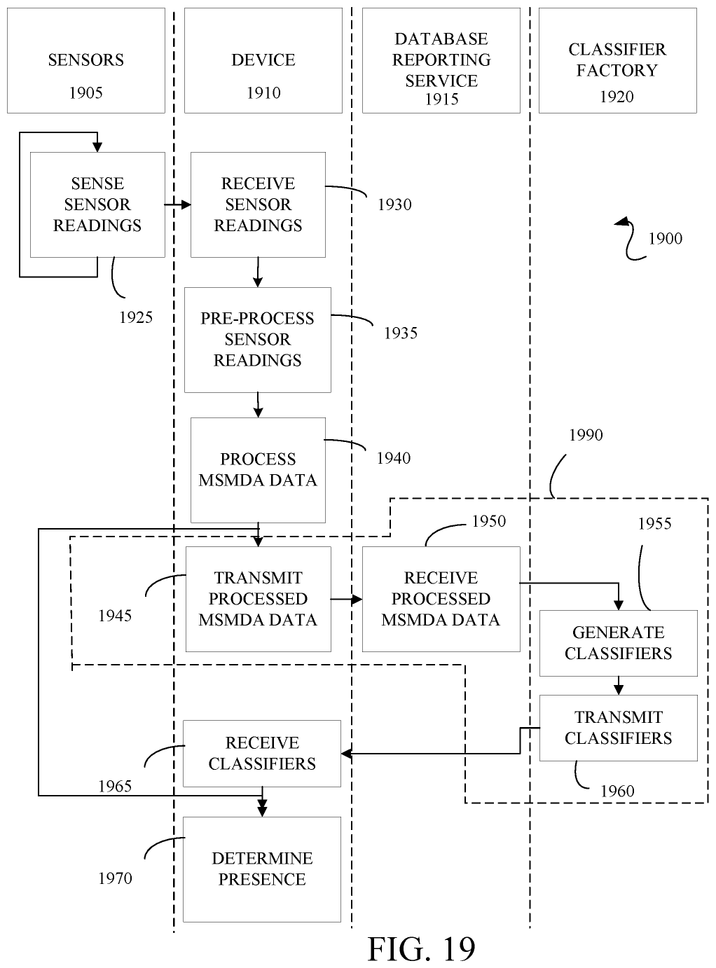

FIG. 19 is a swim lane diagram 1900 for detecting bed presence for each subject and/or object using machine learning. The swim lane diagram 1900 includes sensors 1905, device(s) 1910, database reporting service 1915, and a classifier factory 1920. In an implementation, the database reporting service 1915 and the classifier factory 1920 can be implemented at computing platform 420, for example. In an implementation, the database reporting service 1915 and the classifier factory 1920 can be implemented at the device 1910, for example.

At training time, sensor readings 1925 from the sensors 1905 are received by the device 1910 (1930). The sensor readings are pre-processed 1935 to generate MSMDA data and then the MSMDA data is processed 1940 (for example, to generate features or to map into the kernel space) as described herein. The device 1910 transmits the processed MSMDA data 1945 to the database reporting service 1915. The database reporting service 1915 receives the processed MSMDA data 1950. The classifier factory 1920 generates classifiers 1955 from the received processed MSMDA data, and annotations (if supervised). The classifier factory 1920 transmits the classifiers 1960 to the device(s) 1910. The device(s) 1910 receive the classifiers 19665 and use the classifiers to determine bed presence 1970. In this instance, transmitting the processed sensor readings 1945, receiving the processed sensor readings 1950, generating classifiers 1955, and transmitting the classifiers 1960 are performed during training time as training blocks 1990.

At operation time, the sensor readings 1925 from the sensors 1905 are received by the device 1910 (1930). The sensor readings are pre-processed 1935 to generate MSMDA data and then the MSMDA data is processed 1940 (for example, to generate features or to map into the kernel space) as described herein and fed into the classifier to determine and classify bed presence 1970.

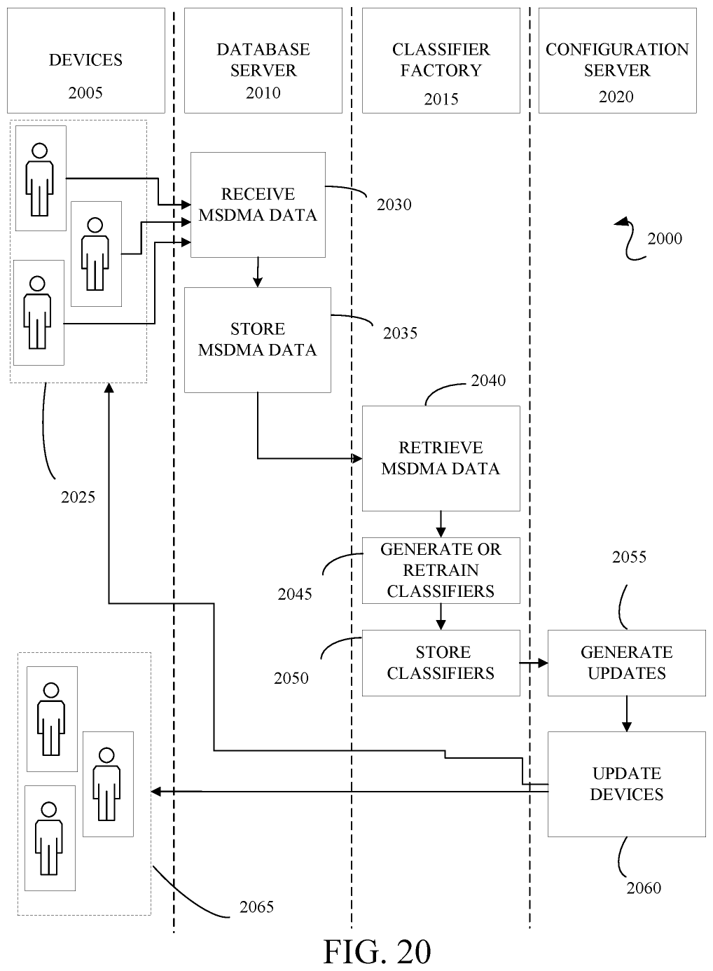

FIG. 20 is a swim lane diagram 2000 for generating classifiers for new devices or refreshing classifiers for existing devices. The swim lane diagram 2000 includes devices 2005 which include a first set of devices 2025 and a second set of devices 2065, a database server 2010, classifier factory 2015, and a configuration server 2020. In an implementation, the database server 2010, the classifier factory 2015, and the configuration server 2020 can be implemented at computing platform 420, for example.

The first set of devices 2025 generate MSMDA data which are received (2030) and stored (2035) by the database server 2010. The classifier factory 2015 retrieves the MSMDA data (2040) and generates or retrains classifiers using the MSMDA data (2045). The generated or retrained classifiers are stored by the classifier factory 2015 (2050). The configuration server 2020 obtains the generated or retrained classifiers and generates an update (2055) for devices 2005. The configuration server 2020 sends the update (2060) to both the first set of devices 2025 and to the second set of devices 2065, where the second set of devices 2065 may be new devices. This system can be used to retrain classifiers on old devices (such as the first set of devices 2025) as more data input is available from more devices 2005. The system can also be used to provide software updates with improved accuracy and can also learn personalized patterns and increase personalization of classifiers or data.

In general, a method for determining item specific parameters includes generating multiple sensor multiple dimensions array (MSMDA) data from multiple sensors, where each of the multiple sensors capture sensor data for one or more items in relation to a substrate, and where an item is a subject or an object. For each identified item, the method includes determining relationships between the multiple sensors based on characteristics of the MSMDA data, determining a location of the item on the substrate based on at least the determined relationships between the multiple sensors, determining an angular orientation of the item on the substrate based on at least the determined relationships between the multiple sensors, and determining a body position of the item on the substrate based at least the determined relationships between the multiple sensors, the location of the subject, and the angular orientation of the item. In an implementation, the method further includes identifying a presence and an order of the presence of each item on the substrate. In an implementation, the method further includes for each item, determining weights based on characteristics of the MSMDA data. In an implementation, the method further includes for each item, comparing the weight against a threshold to determine a bed presence status for the item, and issuing a bed presence status if the weight is greater than the threshold. In an implementation, the threshold is multiple thresholds and each threshold of the multiple thresholds is different for subsequent items. In an implementation, the method further includes for each item, adjusting each threshold based on the location, angular orientation, and body position of for each identified item. In an implementation, the method further includes for each item, adjusting a threshold based on the location, angular orientation, and body position of each identified item, performing a motion analysis based on characteristics of the MSMDA data, and determining a bed presence status for the item based on the weight, the adjusted threshold, and the motion analysis. In an implementation, the determining relationships further includes for each item determining, for a given combination of the multiple sensors: an amplitude change from the MSMDA data; a rate of change from the MSMDA data; a phase change from the MSMDA data; a spectral change from the MSMDA data; a time-frequency change from the MSMDA data; sorting the combinations based on defined metrics, and identifying, for each sorted combination, a determined relationship by: assigning a positive value to any other sorted combination which has at least one of a similar amplitude change, similar rate of change, similar phase of change, similar spectral change, or similar time-frequency change, and assigning a negative value to any other sorted combination which has at least one of an opposite amplitude change, opposite rate of change, or opposite phase of change, opposite spectral change, or opposite time-frequency change, wherein each determined relationship is a pair of combinations. In an implementation, the determining a location further includes for each item identifying determined relationships having one of a same directional change or an opposite directional change, selecting directionally related determined relationships which represent a defined surface coverage area, and mapping the selected directionally related determined relationships to a surface location map to determine the location of the identified item. In an implementation, the determining an orientation further includes for each item identifying determined relationships having strongest amplitude and opposite phase, selecting identified determined relationships which represent corners of a defined surface coverage area, and mapping the selected identified determined relationships to an orientation map to determine the orientation of the identified item. In an implementation, the determining the body position further includes for each item identifying determined relationships having same directional change or an opposite directional change at the location of the identified item and the angular orientation of the identified item, and checking the identified determined relationships against a defined body position to determine the body position of the identified item. In an implementation, the method further includes training a classifier based on the MSMDA data to generate at least a location classifier, an angular orientation classifier, and a body position classifier, and making classifications on non-classified MSMDA data using at least the location classifier, the angular orientation classifier, and the body position classifier. In an implementation, the method further includes updating classifiers associated with other multiple sensors with at least the location classifier, the angular orientation classifier, and the body position classifier, wherein the other multiple sensors and the multiple sensors are associated with different substrates.

In general, a device includes a substrate configured to support an item, where the item is a subject or an object, a plurality of sensors configured to capture sensor data from item actions with respect to the substrate, and a processor in connection with the plurality of sensors. The processor configured to generate multiple sensor multiple dimensions array (MSMDA) data from sensed sensor data, and for each identified item: determine relationships between the plurality of sensors based on characteristics of the MSMDA data, determine a location of the identified item on the substrate based on at least the determined relationships between the plurality of sensors, determine an angular orientation of the identified item on the substrate based on at least the determined relationships between the plurality of sensors, and determine a body position of the identified item on the substrate based at least the determined relationships between the plurality of sensors, the location of the identified item and the angular orientation of the identified item. In an implementation, the processor further configured to identify a presence of each item and the order of the presence on the substrate. In an implementation, the processor further configured to, for each item: determine a weight based on characteristics of the MSMDA data, compare the weight against threshold to determine a bed presence status for the identified item, and issue a bed presence status if the weight is greater than the threshold. In an implementation, the threshold is multiple thresholds and each threshold of the multiple thresholds is different for subsequent items. In an implementation, the processor further configured to, for each item adjust the threshold based on the location of the identified item. In an implementation, the processor further configured to, for each item:determine a weight based on characteristics of the MSMDA data, adjust a threshold based on the location of the identified item, perform a motion analysis based on characteristics of the MSMDA data, and determine a bed presence status for the identified item based on the weight, the adjusted threshold, and the motion analysis. In an implementation, the processor further configured to, for each item, determine, for a given combination of the plurality of sensors: an amplitude change from the MSMDA data; a rate of change from the MSMDA data; a phase change from the MSMDA data; a spectral change from the MSMDA data; a time-frequency change from the MSMDA data, sort the combinations based on a defined metric, and identify, for each sorted combination, a determined relationship by: assignment of a positive value to any other sorted combination which has at least one of a similar amplitude change, similar rate of change, or similar phase of change; and assignment of a negative value to any other sorted combination which has at least one of an opposite amplitude change, opposite rate of change, or opposite phase of change, wherein each determined relationship is a pair of combinations. In an implementation, the processor further configured to, for each item: identify determined relationships having one of a same directional change or an opposite directional change; select directionally related determined relationships which represent a defined surface coverage area; and map the selected directionally related determined relationships to a surface location map to determine the location of the identified item. In an implementation, the processor further configured to, for each item identify determined relationships having strongest amplitude and opposite phase; select identified determined relationships which represent corners of a defined surface coverage area; and map the selected identified determined relationships to an orientation map to determine the angular orientation of the identified item. In an implementation, the processor further configured to, for each item: identify determined relationships having same directional change or an opposite directional change at the location of the identified item and the angular orientation of the identified item; and check the identified determined relationships against a defined body position to determine the body position of the identified item. In an implementation, the device further including a classifier configured to make classifications on non-classified MSMDA data using at least a location classifier, an angular orientation classifier, and a body position classifier, where each of the location classifier, the angular orientation classifier, and the body position classifier is trained and generated based on the MSMDA data.

Implementations of controller 200, controller 214, processor 422, and/or controller 414 (and the algorithms, methods, instructions, etc., stored thereon and/or executed thereby) can be realized in hardware, software, or any combination thereof. The hardware can include, for example, computers, intellectual property (IP) cores, application-specific integrated circuits (ASICs), programmable logic arrays, optical processors, programmable logic controllers, microcode, microcontrollers, servers, microprocessors, digital signal processors or any other suitable circuit. In the claims, the term “controller” should be understood as encompassing any of the foregoing hardware, either singly or in combination.

Further, in one aspect, for example, controller 200, controller 214, processor 422, and/or controller 414 can be implemented using a general purpose computer or general purpose processor with a computer program that, when executed, carries out any of the respective methods, algorithms and/or instructions described herein. In addition or alternatively, for example, a special purpose computer/processor can be utilized which can contain other hardware for carrying out any of the methods, algorithms, or instructions described herein.

Controller 200, controller 214, processor 422, and/or controller 414 can be one or multiple special purpose processors, digital signal processors, microprocessors, controllers, microcontrollers, application processors, central processing units (CPU)s, graphics processing units (GPU)s, digital signal processors (DSP)s, application specific integrated circuits (ASIC)s, field programmable gate arrays, any other type or combination of integrated circuits, state machines, or any combination thereof in a distributed, centralized, cloud-based architecture, and/or combinations thereof.

The word “example,” “aspect,” or “embodiment” is used herein to mean serving as an example, instance, or illustration. Any aspect or design described herein as using one or more of these words is not necessarily to be construed as preferred or advantageous over other aspects or designs. Rather, use of the word “example,” “aspect,” or “embodiment” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

1. A method for determining item specific parameters, the method comprising:
generating multiple sensor multiple dimensions array (MSMDA) data from multiple sensors, wherein each of the multiple sensors capture sensor data for one or more items in relation to a substrate, and wherein an item is a subject or an object;
for each identified item:
determining relationships between the multiple sensors based on characteristics of the MSMDA data;
determining a location of the item on the substrate based on at least the determined relationships between the multiple sensors;
determining an angular orientation of the item on the substrate based on at least the determined relationships between the multiple sensors; and
determining a body position of the item on the substrate based at least the determined relationships between the multiple sensors, the location of the subject, and the angular orientation of the item.

2. The method of claim 1, further comprising:
identifying a presence and an order of the presence of each item on the substrate.

3. The method of claim 1, further comprising, for each item:
determining weights based on characteristics of the MSMDA data.

4. The method of claim 3, further comprising, for each item:
comparing the weight against a threshold to determine a bed presence status for the item; and
issuing a bed presence status if the weight is greater than the threshold.

5. The method of claim 4, wherein the threshold is multiple thresholds and each threshold of the multiple thresholds is different for subsequent items.

6. The method of claim 5, further comprising, for each item:
adjusting each threshold based on the location, angular orientation, and body position of for each identified item.

7. The method of claim 3, further comprising, for each item:
adjusting a threshold based on the location, angular orientation, and body position of each identified item;
performing a motion analysis based on characteristics of the MSMDA data; and
determining a bed presence status for the item based on the weight, the adjusted threshold, and the motion analysis.

8. The method of claim 1, wherein the determining relationships further comprising:
for each item:
determining, for a given combination of the multiple sensors:
an amplitude change from the MSMDA data;
a rate of change from the MSMDA data;
a phase change from the MSMDA data;
a spectral change from the MSMDA data;
a time-frequency change from the MSMDA data;
sorting the combinations based on defined metrics; and
identifying, for each sorted combination, a determined relationship by:
assigning a positive value to any other sorted combination which has at least one of a similar amplitude change, similar rate of change, similar phase of change, similar spectral change, or similar time-frequency change; and
assigning a negative value to any other sorted combination which has at least one of an opposite amplitude change, opposite rate of change, or opposite phase of change, opposite spectral change, or opposite time-frequency change,
wherein each determined relationship is a pair of combinations.

9. The method of claim 8, wherein the determining a location further comprising:
for each item:
identifying determined relationships having one of a same directional change or an opposite directional change;
selecting directionally related determined relationships which represent a defined surface coverage area; and
mapping the selected directionally related determined relationships to a surface location map to determine the location of the identified item.

10. The method of claim 9, wherein the determining an orientation further comprising:
for each item:
identifying determined relationships having strongest amplitude and opposite phase;
selecting identified determined relationships which represent corners of a defined surface coverage area; and
mapping the selected identified determined relationships to an orientation map to determine the orientation of the identified item.

11. The method of claim 8, wherein the determining the body position further comprising:
for each item:
identifying determined relationships having same directional change or an opposite directional change at the location of the identified item and the angular orientation of the identified item; and
checking the identified determined relationships against a defined body position to determine the body position of the identified item.

12. The method of claim 1, further comprising:
training a classifier based on the MSMDA data to generate at least a location classifier, an angular orientation classifier, and a body position classifier; and
making classifications on non-classified MSMDA data using at least the location classifier, the angular orientation classifier, and the body position classifier.

13. The method of claim 12, further comprising:
updating classifiers associated with other multiple sensors with at least the location classifier, the angular orientation classifier, and the body position classifier,
wherein the other multiple sensors and the multiple sensors are associated with different substrates.

14. A device comprising:
a substrate configured to support an item, wherein the item is a subject or an object;
a plurality of sensors configured to capture sensor data from item actions with respect to the substrate;
a processor in connection with the plurality of sensors, the processor configured to:
generate multiple sensor multiple dimensions array (MSMDA) data from sensed sensor data;
for each identified item:
determine relationships between the plurality of sensors based on characteristics of the MSMDA data;
determine a location of the identified item on the substrate based on at least the determined relationships between the plurality of sensors;
determine an angular orientation of the identified item on the substrate based on at least the determined relationships between the plurality of sensors; and
determine a body position of the identified item on the substrate based at least the determined relationships between the plurality of sensors, the location of the identified item and the angular orientation of the identified item.

15. The device of claim 14, the processor further configured to:
identify a presence of each item and the order of the presence on the substrate.

16. The device of claim 14, the processor further configured to:
for each item:
determine a weight based on characteristics of the MSMDA data;
compare the weight against threshold to determine a bed presence status for the identified item; and
issue a bed presence status if the weight is greater than the threshold.

17. The method of claim 16, wherein the threshold is multiple thresholds and each threshold of the multiple thresholds is different for subsequent items.

18. The device of claim 16, the processor further configured to:
for each item:
adjust the threshold based on the location of the identified item.

19. The device of claim 14, the processor further configured to:
for each item:
determine a weight based on characteristics of the MSMDA data;
adjust a threshold based on the location of the identified item;
perform a motion analysis based on characteristics of the MSMDA data; and
determine a bed presence status for the identified item based on the weight, the adjusted threshold, and the motion analysis.

20. The device of claim 14, the processor further configured to:
for each item:
determine, for a given combination of the plurality of sensors:
an amplitude change from the MSMDA data;
a rate of change from the MSMDA data;
a phase change from the MSMDA data;
a spectral change from the MSMDA data;
a time-frequency change from the MSMDA data;
sort the combinations based on a defined metric; and
identify, for each sorted combination, a determined relationship by:
assignment of a positive value to any other sorted combination which has at least one of a similar amplitude change, similar rate of change, or similar phase of change; and
assignment of a negative value to any other sorted combination which has at least one of an opposite amplitude change, opposite rate of change, or opposite phase of change,
wherein each determined relationship is a pair of combinations.

21-24. (canceled)
